SEQUENCE LISTING (15 sequences)

SEQ ID NO: 1 - DNA, 35209 bp, Adenovirus
  Gene (2705)..(4342): yCD/mutTKSR39 Fusion Gene
tcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcatcgaagatccatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggcttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtataggtggcttaactgtatagatctaattccctggcattatgcccagtacatgaccttatgggactttcctacttggcagtacatctacgtattagtcatcgctattaccatggtgatgcggttttggcagtacatcaatgggcgtggatagcggtttgactcacggggatttccaagtcgccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctggggatcttcgagtcgagggatccctcgagccaccatggggacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgagggtggagggggaggcggtgggggaggtgctagcatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccatcttcctcgaccgccatcccatcgccttcatgctgtgctacccggccgcgcggtaccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactgaaacacggaagaattcaagcttgtcgacttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgaagatctggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatcctctagttaatactagtatttaaataattccctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctggggatcagtcttcgagtcgaggatcccctgctccagagatgaccggctcaaccatcgcgcccacaacggactatcgcaacaccactgctaccggactaacatctgccctaaatttaccccaagttcatgcctttgtcaatgactgggcgagcttggacatgtggtggttttccatagcgcttatgtttgtttgccttattattatgtggcttatttgttgcctaaagcgcagacgcgccagaccccccatctataggcctatcattgtgctcaacccacacaatgaaaaaattcatagattggacggtctgaaaccatgttctcttcttttacagtatgattaaatgagacatgattccaagcttgtcgacttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgattaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgat

SEQ ID NO: 2 - DNA, 5100 bp, Adenovirus
tcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcatcgaagatccatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtataggtggcttaactgtatagatctaattccctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctggggatcttcgagtcgagggatccctcgagccaccatggggacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgagggtggagggggaggcggtgggggaggtgctagcatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccatcttcctcgaccgccatcccatcgccttcatgctgtgctacccggccgcgcggtaccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactgaaacacggaagaattcaagcttgtcgacttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgaagatctggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtct

SEQ ID NO: 3 - DNA, 917 bp, Adenovirus
atttaaataattccctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctggggatcagtcttcgagtcgaggatcccctgctccagagatgaccggctcaaccatcgcgcccacaacggactatcgcaacaccactgctaccggactaacatctgccctaaatttaccccaagttcatgcctttgtcaatgactgggcgagcttggacatgtggtggttttccatagcgcttatgtttgtttgccttattattatgtggcttatttgttgcctaaagcgcagacgcgccagaccccccatctataggcctatcattgtgctcaacccacacaatgaaaaaattcatagattggacggtctgaaaccatgttctcttcttttacagtatgattaaatgagacatgattccaagcttgtcgacttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgattaattaa

SEQ ID NO: 4 - DNA, 1635 bp, Adenovirus
  CDS (1)..(1635)
atggggacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgagggtggagggggaggcggtgggggaggtgctagcatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccatcttcctcgaccgccatcccatcgccttcatgctgtgctacccggccgcgcggtaccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaac

SEQ ID NO: 5 - DNA, 303 bp, Adenovirus
  CDS (1)..(303)
atgaccggctcaaccatcgcgcccacaacggactatcgcaacaccactgctaccggactaacatctgccctaaatttaccccaagttcatgcctttgtcaatgactgggcgagcttggacatgtggtggttttccatagcgcttatgtttgtttgccttattattatgtggcttatttgttgcctaaagcgcagacgcgccagaccccccatctataggcctatcattgtgctcaacccacacaatgaaaaaattcatagattggacggtctgaaaccatgttctcttcttttacagtatgat

SEQ ID NO: 6 - protein, 545 aa, Adenovirus
MGTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGGGGGGGGGASMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTIFLDRHPIAFMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 7 - protein, 101 aa, Adenovirus
MTGSTIAPTTDYRNTTATGLTSALNLPQVHAFVNDWASLDMWWFSIALMFVCLIIMWLICCLKRRRARPPIYRPIIVLNPHNEKIHRLDGLKPCSLLLQYD

SEQ ID NO: 8 - DNA, 42 bp, Artificial Sequence
  DNA Primer
gatcggatccctcgagatcctagcatggcttcgtaccccggc

SEQ ID NO: 9 - DNA, 31 bp, Artificial Sequence
  DNA Primer
gatcgaattcttccgtgtttcagttagcctc

SEQ ID NO: 10 - DNA, 33 bp, Artificial Sequence
  DNA Primer
gatcctcgagccaccatggtgacagggggaatg

SEQ ID NO: 11 - DNA, 52 bp, Artificial Sequence
  DNA Primer
gatcgctagcacctcccccaccgcctctccctccaccctcaccaatatcttc

SEQ ID NO: 12 - DNA, 31 bp, Artificial Sequence
  DNA Primer
gatcggatcccctgctccagagatgaccggc

SEQ ID NO: 13 - DNA, 29 bp, Artificial Sequence
  DNA Primer
gatcaagcttggaatcatgtctcamaatc

SEQ ID NO: 14 - DNA, 36 bp, Artificial Sequence
  DNA Primer
gatcatttaaataattccctggcattatgcccagta

SEQ ID NO: 15 - DNA, 36 bp, Artificial Sequence
  DNA Primer
gatcttaattaatcgatgctagacgatccagacatg

(19) United States
(12) Patent Application Publication
(10) Pub. No.: US 2009/0285783 A1
(43) Pub. Date: Nov. 19, 2009

(54) Methods and compositions for cancer therapy using a novel adenovirus

(75) Inventors: Svend D. Freytag, West Bloomfield, MI (US); Jae Ho Kim, West Bloomfield, MI (US); Ken Barton, Sterling Heights, MI (US); Dell Paielli, Wyandotte, MI (US)

Correspondence Address: RADER, FISHMAN & GRAUER PLLC, 39533 WOODWARD AVENUE, SUITE 140, BLOOMFIELD HILLS, MI, 48304-0610, US

(73) Assignee: Henry Ford Health System

(21) Appl. No.: 10/888,492

(22) Filed: Jul. 9, 2004

Related U.S. Application Data
(60) Provisional application No. 60/486,219, filed on Jul. 9, 2003.

(30) Foreign Application Priority Data
Jul. 9, 2004 (US) PCT/US04/22320

(51) Int. Cl. A61K 35/76; A61P 35/00; C07D 239/22; C07D 487/04; C07K 14/435; C12N 15/57; C12N 7/01

(52) U.S. Cl. 424/93.2; 435/235.1; 530/350; 536/23.2; 544/276; 544/317

(57) ABSTRACT

The invention comprises a novel virus that can kill mammalian cancer cells efficiently. The virus produces a novel protein that converts two non-toxic prodrugs into potent chemotherapeutic agents. These chemotherapeutic agents are produced locally and help the virus kill the cancer cells as well as sensitize them to radiation. In preclinical studies, the virus has proven effective at killing a variety of mammalian cancer cells either alone or when combined with prodrug therapy and/or radiation therapy. The invention may provide a safe and effective treatment for human cancer.

### CROSS-REFERENCE TO RELATED APPLICATIONS

This application claims priority based on U.S. Provisional Patent Application No. 60/486,219, filed Jul. 9, 2003, which is hereby incorporated by reference in full.

### FIELD OF THE INVENTION

Generally, the present invention relates to a cancer therapy. More specifically, the present invention relates to an adenovirus-based cancer therapy.

### BACKGROUND

Despite advances in both diagnosis and therapy, the annual number of cancer related deaths has not decreased during the past 60 years. Although conventional cancer therapies (surgery, radiotherapy, chemotherapy) produce a high rate of cure for patients with early stage disease, many cancers recur and the majority of patients with advanced cancer eventually succumb to the disease. The limitations of conventional cancer therapies do not derive from their inability to ablate tumor, but rather from limits on their ability to do so without excessively damaging the patient. It is this consideration that constrains the extent of surgical resection, the dose of radiation and volume to be irradiated, and the dose and combination of chemotherapeutic drugs. Improving the effectiveness of a treatment is of no clinical value if there is no significant increase in the differential response between tumor and normal tissue (i.e., therapeutic index).

Nonetheless, improved methods and novel agents for treating cancer have resulted in increased survival time and survival rate for patients with various types of cancer. For example, improved surgical and radiotherapeutic procedures result in more effective removal of localized tumors. Surgical methods, however, can be limited due, for example, to the location of a tumor or to dissemination of metastatic tumor cells. Radiotherapy also can be limited by other factors that limit the dose that can be administered. Tumors that are relatively radioresistant will not be cured at such a dose.

Although a single treatment modality such as radiation therapy, chemotherapy, surgery or immunotherapy can result in improvement of a patient, superior results can be achieved when such modalities are used in combination. In particular, treatment with a combination of radiotherapy, which can be directed to a localized area containing a tumor, and chemotherapy or immunotherapy, which provide a systemic mode of treatment, can be useful where dissemination of the disease has occurred or is likely to occur. Unfortunately, the therapeutic usefulness of radiation therapy can be limited where the tumor cells are relatively radioresistant, since the dose is limited by the tolerance of normal tissue in the radiation field. Thus, there exists a need to sensitize cancer tumors to the effects of radiotherapy so that it can more effectively reduce the severity of a tumor in a patient. Further, it would be useful to develop a treatment that more specifically isolates the location of the radiation, thus preventing the effects of radiation treatment on healthy cells.

In related fashion, to mitigate unwanted effects of some chemotherapies, adenovirus vectors have been used to transduce tumor cells with so-called “chemogenes” that convert a nontoxic substance, or “prodrug”, into a toxic, therapeutically effective form. Several new approaches involving gene therapy are under consideration for improving the therapeutic index of cancer therapies.

One of these approaches, so-called “suicide gene therapy,” involves the transfer and expression of non-mammalian genes encoding enzymes that convert non-toxic prodrugs into toxic anti-metabolites. Two “suicide genes” that are currently being evaluated in clinical trials are the E. coli cytosine deaminase (CD) and herpes simplex virus type-1 thymidine kinase (HSV-1 TK) genes, which confer sensitivity to 5-fluorocytosine (5-FC) and ganciclovir (GCV), respectively. Following targeted transfer of these genes to the tumor, the 5-FC and GCV prodrugs are converted locally into potent chemotherapeutic agents resulting in significant tumor cell death (see reference 1 (and the references cited therein) in the List of References Section below). Thus, the dose-limiting systemic toxicity associated with conventional chemotherapies is mitigated.

Previously, the bacterial CD and wild-type HSV-1 TK genes have been coupled to create a novel CD/HSV-1 TK fusion gene (see reference (hereinafter “ref.”) 1 in the List of References Section). The CD/HSV-1 TK fusion gene allows for combined use of CD/5-FC and HSV-1 TK/GCV suicide gene therapies. It has been previously demonstrated that CD/5-FC and HSV-1 TK/GCV suicide gene therapies render malignant cells sensitive to specific pharmacological agents and importantly, sensitize them to radiation (see refs. 1-9). Using a novel, oncolytic, replication-competent adenovirus (Ad5-CD/TKrep) containing the prototype CD/HSV-1 TK fusion gene (ref. 10), the safety and efficacy of replication-competent adenovirus-mediated double suicide gene therapy without and with radiation therapy in several preclinical cancer models (refs. 10-13) and more recently, in human prostate cancer patients (refs. 14 and 15) have been demonstrated.

In these clinical trials targeting human prostate cancer, the Ad5-CD/TKrep virus proved to be safe up to a dose of 10¹² Vp when combined with up to 3 weeks of 5-FC and GCV (vGCV) prodrug therapy without (ref. 14) and with (ref. 15) conventional dose (70 Gy) three dimensional conformal radiation therapy (3DCRT). Moreover, these treatment regimens have demonstrated signs of clinical activity (refs 14 and 15).

Nonetheless, despite these advances, a significant need remains for inventions that comprise effective methods and compositions for use in cancer therapies. The present invention was developed in light of these and other drawbacks.

### SUMMARY OF THE INVENTION

The present invention comprises novel, improved methods and compositions for cancer therapy which comprise a novel virus that can kill mammalian cancer cells efficiently. The virus produces a novel protein that converts non-toxic prodrugs into potent chemotherapeutic agents. These chemotherapeutic agents are produced locally and help the virus kill the cancer cells as well as sensitize them to radiation. In preclinical studies, the virus has proven effective at killing a variety of human cancer cells either alone or when combined with prodrug therapy and/or radiation therapy.

The invention comprises a novel, “second-generation” adenovirus (designated “Ad5-yCD/mutTK_{SR39}rep-ADP”) with at least two significant improvements relative to the previously disclosed prototype Ad5-CD/TKrep virus. Ad5-yCD/mutTK_{SR39}rep-ADP contains an improved yCD/mutTK_{SR39} fusion gene whose product is more efficient at converting the 5-FC and GCV prodrugs into their active chemotherapeutic agents. Moreover, Ad5-yCD/mutTK_{SR39}rep-ADP expresses the Ad5 ADP protein, which significantly increases the oncolytic activity of replication-competent adenoviruses. Relative to the prototype Ad5-CDITKrep virus, Ad5-yCD/mutTK_{SR39}rep-ADP has demonstrated greater viral oncolytic and chemotherapeutic activity in preclinical cancer models. The data suggest that the Ad5-yCD/mutTK_{SR39}rep-ADP virus comprising the present invention will exhibit low toxicity and significant anti-tumor activity clinically when combined with 5-FC and GCV prodrug therapy and radiation therapy.

Other aspects of the invention will be apparent to those skilled in the art after reviewing the drawings and the detailed description below.

### BRIEF DESCRIPTION OF THE DRAWINGS

The present invention will now be described, by way of example, with reference to the accompanying drawings, in which:

FIG. 1 is a schematic representation of the Ad5-yCD/mutTK_{SR39}rep-ADP virus of the present invention.

FIG. 2 is a diagram showing an advantage of the ADP gene of the present invention.

FIGS. 3A and 3B are diagrams showing the advantage of the improved yCD/mutTK_{SR39} gene of the invention.

FIG. 4 is a diagram showing an advantage of the ADP gene of the present invention

FIG. 5 shows Kaplan-Meier plots with Ad5-yCD/mutTK_{SR39}rep-ADP in intraprostatic LNCaP C4-2 mouse model.

### DETAILED DESCRIPTION OF THE INVENTION

Generally, the present invention comprises methods and compositions for the treatment for cancer. More specifically, the present invention provides a treatment that, when administered with prodrugs, can kill cancer cells and make the remaining cancer cells more sensitive to radiation.

Embodiments of the present invention include a novel virus that produces a protein that can convert non-toxic prodrugs into chemotherapeutic agents. The prodrugs can be produced locally or administered in conjunction with the treatment. Preferably, the virus is an oncolytic, replication-competent adenovirus such as, but not limited to, Ad5-yCD/mutTK_{SR39}rep-ADP. When administered to a patient in need of such treatment, the adenovirus converts at least two prodrugs into chemotherapeutic agents. These prodrugs can include, but are not limited to, 5-fluorocytosine (5-FC) and ganciclovir (GCV and derivatives thereof).

In addition to the ability to convert the prodrugs into chemotherapeutic agents, embodiments of the present invention sensitize the cells to radiation. By sensitizing the cells, lower doses of radiation can be used without limiting the benefits of radiation. Further, the radiation therapy is more effective because the cancer cells are more sensitive to the radiation, while normal cells are not more sensitive, thus limiting the side effects of cancer treatments. The treatment of the present invention can be used in conjunction with other therapies such as surgery, chemotherapy, hormone therapy, and immunotherapy.

In preferred embodiments, the present invention comprises a novel, oncolytic, replication-competent adenovirus (Ad5-yCD/mutTK_{SR39}rep-ADP) containing a yeast cytosine deaminase (yCD)/mutant SR39 herpes simplex virus type-1 thymidine kinase (mutTK_{SR39}) fusion gene and the adenovirus type 5 (Ad5) adenovirus death protein (ADP) gene. Ad5-yCD/mutTK_{SR39}rep-ADP replicates in and kills human cancer cells efficiently. Ad5-yCD/mutTK_{SR39}rep-ADP produces a novel yCD/mutTK_{SR39} fusion protein that can convert two prodrugs, 5-fluorocytosine (5-FC) and ganciclovir (GCV; and GCV derivatives), into potent chemotherapeutic agents (referred to as double suicide gene therapy). Both yCD/5-FC and HSV-1 TK_{SR39} suicide gene therapies exhibit potent chemotherapeutic activity and sensitize tumor cells to ionizing radiation.

By way of example only, preclinical studies show that the Ad5-yCD/mutTK_{SR39}rep-ADP virus is effective at killing a variety of human cancer cells when used by itself or when combined with double suicide gene therapy and/or radiation therapy. In a clinical setting, the Ad5-yCD/mutTK_{SR39}rep-ADP virus could be used as a monotherapy for its virus-mediated oncolytic effect, it could be coupled with yCD/5-FC and HSV-1 Ad5-TK_{SR39}/GCV suicide gene therapies for a combined viral oncolytic/chemotherapeutic effect, or it could be coupled with yCD/5-FC and HSV-1 TK_{SR39}/GCV suicide gene therapies and radiation therapy for a combined viral oncolytic/chemotherapeutic/radiosensitization effect (referred to as trimodal therapy). Trimodal therapy could be combined with other conventional cancer treatments such as surgery, chemotherapy, hormone therapy and immunotherapy in the management of human cancer.

To develop further this gene therapy-based approach as a cancer treatment, a novel, second-generation adenovirus (Ad5-yCD/mutTK_{SR39}rep-ADP) has been developed with two significant improvements relative to the prototype Ad5-CD/TKrep virus. Ad5-yCD/mutTK_{SR39}rep-ADP contains an improved yCD/mutTK_{SR39} fusion gene whose product is more efficient at converting the 5-FC and GCV prodrugs into their active chemotherapeutic agents. Moreover, Ad5-yCD/mutTK_{SR39}rep-ADP expresses the Ad5 ADP protein, which significantly increases the oncolytic activity of replication-competent adenoviruses. Relative to the prototype Ad5-CDITKrep virus, Ad5-yCD/mutTK_{SR39}rep-ADP has demonstrated greater viral oncolytic and chemotherapeutic activity in preclinical cancer models.

Introduction of nucleic acid of the present invention by viral infection offers several advantages over the other listed methods. Higher efficiency can be obtained due to virus' infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

Also, additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation will not occur.

Features that limit expression to particular cell types can also be included in some embodiments. Such features include, for example, promoter and regulatory elements that are specific for the desired cell type.

In addition, recombinant viral vectors are useful for in vivo expression of the nucleic acids of the present invention because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention will depend on desired cell type to be targeted and will be known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

The recombinant vector can be administered in several ways. For example, the procedure can take advantage of the target specificity of viral vectors and consequently do not have to be administered locally at the diseased site. However, local administration can provide a quicker and more effective treatment. Administration can also be performed by, for example, intravenous or subcutaneous injection into the subject. Following injection, the viral vectors will circulate until they recognize host cells with the appropriate target specificity for infection.

An alternate mode of administration can be by direct inoculation locally at the site of the disease or pathological condition or by inoculation into the vascular system supplying the site with nutrients. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes can also be used to introduce the non-viral vectors described above into recipient cells within the inoculated area. Such transfection vehicles are known by one skilled within the art.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including humans. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness. The doses may be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Many other such implants, delivery systems, and modules are well known to those skilled in the art.

In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered will vary for the patient being treated.

### DEFINITIONS

Unless stated otherwise or suggested by context, the following terms and phrases have the meaning provided below.

The term “gene therapy” as used herein refers to the transfer of genetic material (e.g. DNA or RNA) of interest into a host to treat or prevent a genetic or acquired disease or condition phenotype. The genetic material of interest encodes a product (e.g. a protein, polypeptide, peptide, functional RNA, antisense) whose production in vivo is desired. For example, the genetic material of interest can encode a hormone, receptor, enzyme, polypeptide or peptide of therapeutic value. The genetic material of interest can also encode a suicide gene. For a review see, in general, the text “Gene Therapy” (Advances in Pharmacology 40, Academic Press, 1997).

The phrase “in vivo gene therapy” refers to when the genetic material to be transferred is introduced into the target cells of the recipient organism in situ, which is within the recipient. After therapy, the genetically altered target cells express the transfected genetic material in situ. Such therapy also includes repairing the gene in situ, if the host gene is defective.

The phrase “gene expression vehicle” refers to any vehicle capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore, as used herein the expression vehicle may, as needed, not include the 5′UTR and/or 3′UTR of the actual gene to be transferred and only include the specific amino acid coding region. The expression vehicle can include a promoter for controlling transcription of the heterologous material and can be either a constitutive or inducible promoter to allow selective transcription. Enhancers that may be required to obtain necessary transcription levels can optionally be included. Enhancers are generally any non-translated DNA sequence which works contiguously with the coding sequence (in cis) to change the basal transcription level dictated by the promoter. The expression vehicle can also include a selection gene.

### EXAMPLES

1. Description of the Ad5-yCD/mutTK_{SR39}rep-ADP Adenovirus

The complete and partial DNA and translated protein sequences of the Ad5-yCD/mutTK_{SR39}rep-ADP adenovirus, yCD/mutTK_{SR39} fusion gene and ADP gene (SEQ ID NOs. 1-5) are disclosed following the List of References Section. The following examples are presented in view of such sequences.

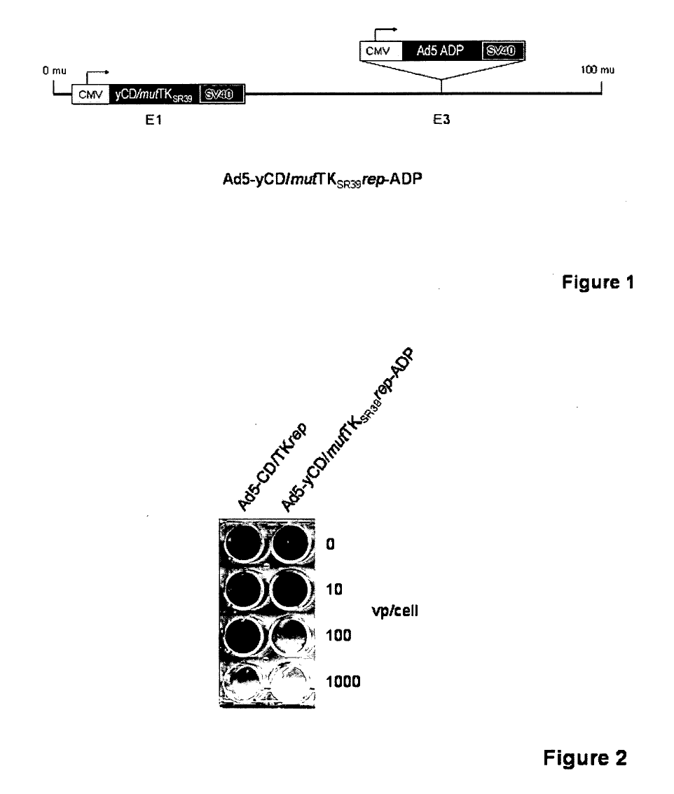

The Ad5-yCD/mutTK_{SR39}rep-ADP virus (SEQ ID NO. 1) of the examples is a replication-competent, type 5 adenovirus (the sequence of which is readily known and obtainable to one skilled in the art) that contains an improved yCD/mutTK_{SR39} fusion gene in the E1 region and the Ad5 ADP gene in the E3 region. A schematic representation of Ad5-yCD/mutTK_{SR39}rep-ADP is provide in FIG. 1 (in FIG. 1, “CMV”=human cytomegalovirus promoter; “SV40”=simian virus 40 polyadenylation sequences; and “mu”=map units.) As shown in FIG. 1, the CMV-yCD/mutTK_{SR39}-SV40 expression cassette is located in the E1 region in place of the deleted 55 kDa E1B gene. The CMV-ADP-SV40 expression cassette is located in the E3 region in place of the deleted E3 genes.

Ad5-yCD/mutTK_{SR39}rep-ADP contains a 1,255 base pair (bp) deletion (bases 2,271 to 3,524) in the 55 kDa E1B gene (see SEQ ID NO. 2). Using methods known to those of ordinary skill in the art, two premature translation stop codons were engineered into the 55 kDa E1B gene resulting in the production of a truncated, non-functional, 78 amino acid E1B protein. Ad5-yCD/mutTK_{SR39}rep-ADP expresses the wild-type Ad5 E1A and 19 kDa E1B proteins. The yCD/mutTK_{SR39} fusion gene (SEQ ID NO. 4) was inserted in place of the deleted 55 kDa E1B gene. Expression of the yCD/mutTK_{SR39} fusion gene is driven by the human cytomegalovirus (CMV) promoter and utilizes simian virus 40 (SV40) polyadenylation elements. The yCD/mutTK_{SR39} fusion gene codes for a 59 kDa yCD/mutTK_{SR39} fusion protein, which is capable of enzymatically converting 5-flurocytosine (5-FC) into fluorouracil (5-FU) and ganciclovir (GCV) and its derivatives into their corresponding monophosphates (e.g. GCV-MP). The downstream metabolic products of 5-FU and GCV-MP are potent inhibitors of DNA replication and result in the death of dividing cells. These downstream metabolic products are also potent radiosensitizers and can markedly increase the therapeutic effect of radiation therapy (see refs. 1-14). Cells that express the yCD/mutTK_{SR39} fusion protein, as well as neighboring cells via the bystander effect, are killed by yCD/5-FC and HSV-1 TK_{SR39}/GCV suicide gene therapies and are sensitized to the killing effects of ionizing radiation.

Ad5-yCD/mutTK_{SR39}rep-ADP also contains a 2.68 kb deletion in the E3 region (bases 28,133 to 30,181), which affects genes that suppress the host immune response but are unnecessary for virus replication (see SEQ ID NO. 3). Ad5-yCD/mutTK_{SR39}rep-ADP contains an Ad5 ADP expression cassette in place of the natural Ad5 E3 genes. Expression of the ADP gene (SEQ ID NO. 5) is driven by the human cytomegalovirus (CMV) promoter and utilizes simian virus 40 (SV40) polyadenylation elements. The authentic 111.6 kDa Ad5 ADP protein is produced, which significantly increases the oncolytic activity of replication-competent adenoviruses. Ad5-yCD/mutTK_{SR39}rep-ADP lacks all other known Ad5 E3 genes (gp19, 10.4 kDa, 14.5 kDa and 14.7 kDa genes).

2. Construction of the Ad5-yCD/mutTK_{SR39}rep-ADP Adenovirus

Plasmids containing adenoviral sequences used in the construction of Ad5-yCD/mutTK_{SR39}rep-ADP were obtained from Microbix (Toronto, Canada). To generate the pCMV-yCD/mutTK_{SR39} expression plasmid (left-end vector), the mutant SR39 HSV-1 TK gene (ref. 16) was generated by the polymerase chain reaction (PCR) using linearized pET23d:HSVTK_{SR39} as template. The following primer pair was used to generate the mutTK_{SR39} PCR product:

5′-GATCGGATCCCTCGAGATC2CTAGCATGGCTTCGTACCCCGGC-3
5′-GATCGAATTCTTCCGTGTTTCAGTTAGCCTC-3

The resulting 1,128 bp fragment was digested with BamHI (GGATCC)+EcoRI (GAATTC) and cloned between the BamHI+EcoRI sites of pCA14-CDglyTK-E1aE1b (ref. 10) after removal of the prototype CD/HSV-1 TK fusion gene generating pCA14-CMV-mutTK_{sR39}-E1aE1b. The yCD gene (ref. 17) was generated by PCR using linearized pBAD-ByCD as template. The following primer pair was used to generate the yCD PCR product:

5′-GATCCTCGAGCCACCATGGTGACAGGGGGAATG-3′
5′-GATCGCTAGCACCTCCCCCACCGCCTCtCCCTCCACCCTCACCAATA
TCTTC-3′

The resulting 526 bp fragment was digested with XhoI (CTCGAG)+NheI (GCTAGC) and cloned between the XhoI+NheI sites of pCA14-CMV-mutTK_{SR39}-E1aE1b generating pCA14-CMV-yCD/mutTK^{SR39-}E1aE1b.

To generate pBHG10-PacImod-CMV-ADP (right-end vector), the ADP gene was generated by PCR and cloned between the PacI and SwaI sites of pBHG10-PacImod. pBHG10-PacImod is a derivative of pBHG10 (Microbix; Toronto, Canada) and contains PacI and SwaI sites in the E3 region to facilitate directional cloning.

pBHG10 is a plasmid that contains the entire adenovirus type 5 genome minus bases 188 to 1,339 in the E1 region and bases 28,133 to 30,818 in the E3 region. Using wild-type Ad5 DNA as template, a 333 bp PCR product containing the ADP gene was generated. The following primer pair was used to generate the ADP PCR product:

5′-GATCGGATCCCCTGCTCCAGAGATGACCGGC.3′
5′-GATCAAGCTTGGAATCATGTCTCAMAATC-3′

The resulting 333 bp PCR product was digested with BamHI (GGATCC)+HindIII (AAGCTT) and cloned into BamHI-HindIII digested pCA14 (Microbix; Toronto, Canada) generating pCA14-ADP. The entire CMV-ADP-SV40 polyA expression cassette was generated by PCR using the following primer pair:

5′-GATCATTTAAATAATTCCCTGGCATTATGCCCAGTA-3′
5′-GATCTTAATTAATCGATGCTAGACGATCCAGACATG-3′

A SwaI restriction site (ATTTAAAT) was introduced upstream of the CMV promoter in the 5′ primer and a PacI restriction site (TTAATTAA) was introduced downstream of the SV40 poly A region with the 3′primer. The PCR product was digested with SwaI and PacI and cloned into SwaI-PacI digested pBGH10-PacImod generating pBGH10-PacImod-CMV-ADP.

To generate Ad5-yCD/mutTK_{SR39}rep-ADP virus, pCA14-CMV-yCD/mutTK_{SR39}-E1aE1b (10 μg) was linearized by PvuI digestion and co-transfected with ClaI-linearized pBHG10-PacImod-CMV-ADP (30 μg) into HEK 293 cells (Microbix) using the CaPO₄-DNA precipitation method. Isolated plaques were harvested 7-14 days later and plaque-purified a second time on HEK 293 cells. Virus form twice purified plaques was used to infect HEK 293 cells to generate crude viral supernatants and CsCl gradient-purified adenovirus.

3. Advantage of the ADP Gene Contained in Ad5-yCD/mutTK_{SR39}rep-ADP in vitro

Human DU145 prostate adenocarcinoma cells were plated in a 24-well plate at a concentration of 5×10⁴ cells/well and were infected with graded amounts of thee Ad5-CD/TKrep (lane 1) and Ad5-yCD/mutTK_{SR39}rep-ADP viruses (lane 2). Five days later, cells were fixed and stained with crystal violet. The results (as shown in FIG. 2, “Vp”=viral particles) clearly demonstrate that replication-competent adenoviruses containing the Ad5 ADP gene and expressing the ADP protein (i.e. Ad5-yCD/mutTK_{SR39}rep-ADP) possess significantly greater oncolytic activity than adenoviruses that lack ADP. In other words, the presence of the Ad5 ADP gene significantly increased the oncolytic activity of replication competent adenoviruses. These results demonstrate, in vitro, the advantage of the ADP gene contained in Ad5-yCD/mutTK_{SR39}rep-ADP.

4. Advantage of the yCD/mutTK_{SR39} Gene Contained in Ad5-yCD/mutTK_{SR39}rep-ADP in vitro

### A. CD Assays

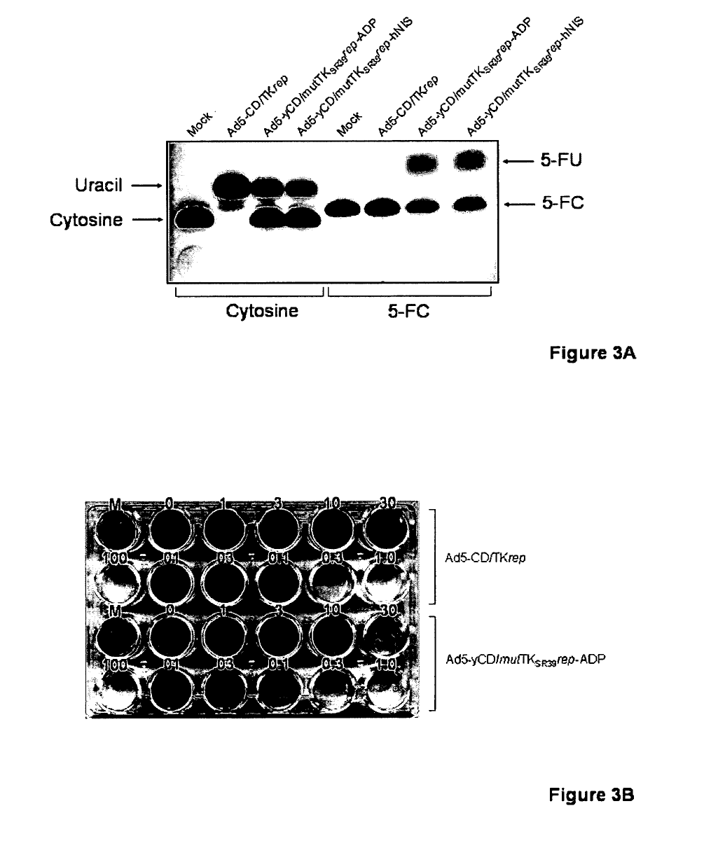

LNCaP C4-2 cells were mock-infected (lanes 1 & 5), or infected with Ad5-CD/TKrep (lanes 2 & 6), Ad5-yCD/mutTK_{SR39}rep-ADP (lanes 3 & 7), Ad5-yCD/mutTK_{SR39} rep-hNIS (lanes 4 & 8) at a MOI of 10. Seventy two hours later, cells were examined for CD activity using [¹⁴C]-cytosine (lanes 1-4) and [³H]-5-FC (lanes 4-8) as substrates. The results are shown in FIG. 3A [(Cytosine (lower left arrow), uracil (upper left arrow), 5-FC (upper right arrow), 5-FU (lower right arrow)]. As shown by FIG. 3A, recombinant adenoviruses that express the improved yCD/mutTK_{SR39}rep gene, such as Ad5-yCD/mutTK_{SR39}rep-ADP, demonstrate greater conversion of 5-FC into 5-FU, but not cytosine into uracil, than viruses expressing the CD/HSV-1 TK fusion gene contained in the prototype Ad5-CD/TKrep virus.

### B. Cytopathic Effect Assay

Cells (10⁶ cells, 60 mm dish) were mock-infected or infected with Ad5-CD/TKrep or Ad5-yCD/mutTK_{SR39}rep-ADP at an MOI of 3. The next day, cells were replated (24 well plate) in medium containing varying concentrations of 5-FC (wells 3-7 and 15-19, going left to right, top to bottom) or GCV (wells 8-12 and 20-24, going left to right, top to bottom) in μg/ml. Cells were stained with crystal violet 9 days later. The results (as shown in FIG. 3B) demonstrate that recombinant adenoviruses expressing the improved yCD/mutTKrep gene, such as Ad5-yCD/mutTK_{SR39}rep-ADP, achieve greater cell kill when combined with 5-FC prodrug therapy than viruses expressing the CD/HSV-1 TK fusion gene contained in the prototype Ad5-yCD/TKrep virus. Together, the results of FIGS. 3A and 3B show, in vitro, the advantage of the yCD/mutTK_{SR39} gene, which is contained in Ad5-yCD/mutTK_{SR39}rep-ADP.

The results of this example also demonstrate that yCD/5-FC and HSV-1 TK_{SR39}/GCV suicide gene therapies can be used to increase the therapeutic effect of the Ad5-yCD/mutTK_{SR39}rep-ADP virus itself. Ad5-yCD/mutTK_{SR39}rep-ADP contains a novel yCD/mutTK_{SR39} fusion gene whose product has improved catalytic activity relative to the CD/HSV-1 TK fusion protein produced by the prototype Ad5-CD/TKrep virus. Recombinant adenoviruses that express the improved yCD/mutTK_{SR39} fusion protein demonstrate significantly greater conversion of 5-FC into 5-FU, and possibly GCV into GCV-MP, than viruses that express the prototype CD/HSV-1-TK fusion protein. Thus, yCD/5-FC and HSV-1 TK_{SR39}/GCV suicide gene therapies can be used independently and together to augment the tumor destructive effects of the Ad5-yCD/mutTK_{SR39}rep-ADP virus.

5. Advantage of the ADP Gene Contained in Ad5-yCD/mutTK_{SR39}rep-ADP in vivo

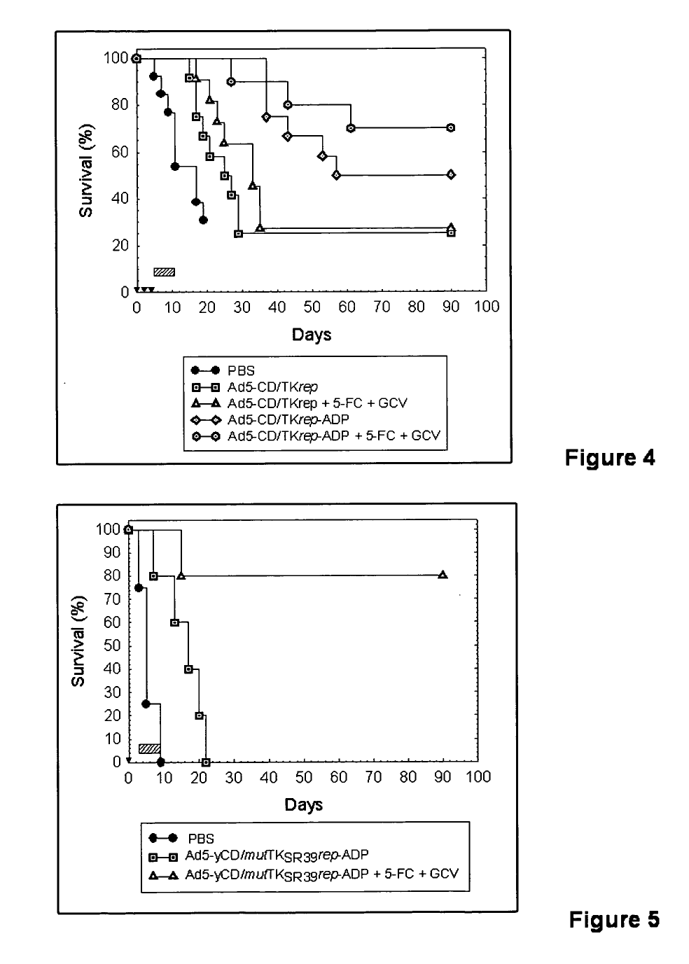

Intramuscular (leg) C33A tumors (150-200 mm³) were injected with 10¹⁰ vp of Ad5-CD/TKrep or Ad5-CD/TKrep-ADP on Days 0, 2 and 4 (arrowheads in FIG. 4). 5-FC (500 mg/kg/day) and GCV (30 mg/kg/day) were administered on Days 5-11 (hatched bar in FIG. 4). Tumor volume was monitored every other day. The predetermined endpoint was 500 mm³. Survival is defined as an animal having no tumor (cure) or a tumor <500 mm³ on Day 90. The results (as shown in FIG. 4 and Table 1 below) show greater destruction of tumor cells in vivo and thus demonstrate the advantage of the ADP gene, which is contained in Ad5-yCD/mutTK_{SR39}rep-ADP. In other words, the presence of the Ad5 ADP gene significantly increased the oncolytic activity of replication competent adenoviruses in vivo as well as in vitro.

TABLE 1
Summary of Results with Ad5-CD/TKrep-ADP in C33A Tumor
Model.

| | | | P value | |
|---|---|---|---|---|
| | | Tumor | | Fisher Exact |
| | Median | Care | Log Rank | (Tumor |
| Group | Survival | (%) | (Survival) | Cure) |
| PBS | 17 | 0 (0/13) | | |
| Ad5-CD/TKrep | 26 | 0 (0/12) | | |
| Ad5-CD/TKrep + 5-FC + | 33 | 9 (1/11) | | |
| GCV | | | | |
| Ad5-CD/TKrep-ADP | >90 | 8 (1/12) | 0.022^{b} | 1.000^{b} |
| Ad5-CD/TKrep-ADP + | >90 | 70 (7/10) | 0.026^{c} | .008^{c} |
| 5-FC + GCV | | | | |

^{a}Median survival is in days.
^{b}Ad5-CD/TKrep-ADP vs. Ad5-CD/TKrep
^{c}Ad5-CD/TKrep-ADP + 5-FC + GCV vs. Ad5-CD/TKrep + 5-FC + GCV

6. Effectiveness of Ad5-yCD/mutTK_{SR39}rep-ADP in vivo in Mouse Model

Male SCID mice bearing intraprostatic LNCaP C4-2 tumors (˜25-50 mm³ in size) were injected with about 10⁹ vp of Ad5-yCD/mutTK_{SR39}rep-ADP on Day 0 (arrowhead in FIG. 5). 5-FC (500 mg/kg/day) and GCV (30 mg/kg/day) were administered on Days 3-9 (hatched bar in FIG. 5). Serum PSA was measured weekly. The predetermined endpoint was PSA=500 ng/ml. The results (as shown in FIG. 5 and Table 2) show an increase in median survival time and/or tumor cure in mouse model using Ad5-yCD/mutTK_{SR39}rep-ADP of the present invention.

TABLE 2
Results with Ad5-yCD/mutTK_{SR39}rep-ADP
in LNCaP C4-2 Tumor Model.

| | | | P value | |
|---|---|---|---|---|
| | | | | Fisher |
| | Median | Tumor | | Exact |
| | Survival | cure | Log Rank | (Tumor |
| Group | (days) | (%) | (Survival) | Cure) |
| PBS | 5 | 0 (0/8) | | |
| Ad5-yCD/mutTK_{SR39}rep-ADP | 17 | 0 (0/11) | .038^{a} | NA^{a} |
| Ad5-yCD/mutTK_{SR39}rep- | >90 | 80 (8/10) | <0.001^{b} | <0.001^{b} |
| ADP + 5-FC + GCV | | | | |

^{a}Ad5-yCD/mutTK_{SR39}rep-ADP vs. PBS;
^{b}Ad5-yCD/mutTK_{SR39}rep-ADP + 5-FC + GCV vs. PBS.

7. Radiosensitized Human Cancer Cells Using yCD/5-FC and HSV-1 TK_{SR39}/GCV

As shown in previous experiments by the inventors (see refs. 1-14), yCD/5-FC and HSV-1 TK_{SR39}/GCV suicide gene therapies can also be used to radiosensitize human cancer cells. Ad5-yCD/mutTK_{SR39}rep-ADP contains a novel yCD/mutTK_{SR39} fusion gene whose product has improved catalytic activity relative to the CD/HSV-1 TK fusion protein produced by the prototype Ad5-CD/TKrep virus. The previous studies demonstrated that CD/5-FC and HSV-1 TK/GCV suicide gene therapies can sensitize human tumor cells to ionizing radiation. Thus, since Ad5-yCD/mutTK_{SR39} rep-ADP expresses an improved yCD/mutTK_{SR39} fusion protein, it may result in greater tumor cell radiosensitization in vivo.

Throughout this application, various references are noted by reference numbers. A numbered list of these references with their full citations is provided below. The disclosures of these references in their entireties are hereby incorporated by reference into this application in order to more fully describe the state of the art to which this invention pertains.

While the present invention has been particularly shown and described with reference to the foregoing preferred and alternative embodiments, and examples, it should be understood by those skilled in the art that various alternatives to the embodiments of the invention described herein may be employed in practicing the invention without departing from the spirit and scope of the invention as defined in the following claims. It is intended that the following claims define the scope of the invention and that the method and composition within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

### LIST OF REFERENCES

1. Rogulski, K. R., Kim, J. H., Kim, S. H., and Freytag, S. O. Glioma cells transduced with an E. coli CD/HSV-1 TK fusion gene exhibit enhanced metabolic suicide and radiosensitivity. Hum. Gene Ther., 8: 73-85, 1997.
2. Kim, J. H., Kim, S. H., Brown, S. L., and Freytag, S. O. Selective enhancement by an antiviral agent of the radiation-induced cell killing of human glioma cells transduced with HSV-tk gene. Cancer Res., 54: 6003-6056, 1994.
3. Kim, J. H., Kim, S. H., Kolozsvary, A., Brown, S. L., Kim, O. B., and Freytag, S. O. Selective enhancement of radiation response of herpes simplex virus thymidine kinase transduced 9L gliosarcoma cells in vitro and in vivo by antiviral agents. Int. J. Radiat. Oncol. Biol. Phys., 33: 861-868, 1995.
4. Khil, M., Kim, J. H., Mullen, C. A., Kim, S. H., and Freytag, S. O. Radiosensitization by 5-fluorocytosine of human colorectal carcinoma cells in culture transduced with cytosine deaminase gene. Clin. Cancer Res., 2: 53-57, 1996.
5. Kim, S. H., Kim, J. H., Kolozsvary, A., Brown, S. L., and Freytag, S. O. Preferential radiosensitization of 9L glioma cells transduced with HSV-TK gene by acyclovir. J. Neurooncol., 33: 189-194, 1997.
6. Gable, M., Kim, J. H., Kolozsvary, A., Khil, M., and Freytag, S. O. Selective in vivo radiosensitization by 5-fluorocytosine of human colorectal carcinoma cells transduced with the E. coli cytosine deaminase gene. Int. J. Radiat. Oncol. Biol. Phys., 41: 883-887, 1998.
7. Rogulski, K. R., Zhang, K., Kolozsvary, A., Kim, J. H., and Freytag, S. O. Pronounced antitumor effects and tumor radiosensitization of double suicide gene therapy. Clin. Cancer Res., 3: 2081-2088, 1997.
8. Kim, J. H., Kolozsvary, A., Rogulski, K. R., Khil, M., and Freytag, S. O. Selective radiosensitization of 9L glioma. in the brain transduced with double suicide fusion gene. Can. J. Scient Am. 4:364-369, 1998.
9. Xie, Y., Gilbert, J. D., Kim, J. H., and Freytag, S. O. Efficacy of adenovirus-mediated CD/5-FC and HSV-1TK/GCV suicide gene therapies concomitant with p53 gene therapy. Clin. Cancer Res., 5: 4224-4232, 1999.
10. Freytag, S. O., Rogulski, K. R., Paielli, D. L., Gilbert, J. D., and Kim, J. H. A novel three-pronged approach to selectively kill cancer cells: concomitant viral, double suicide gene, and radiotherapy. Hum. Gene Ther., 9: 1323-1333, 1998.
11. Rogulski, K. R., Wing, M., Paielll, D. L., Gilbert, J. D., Kim, J. H., and Freytag, S. O. Double suicide gene therapy augments the antitumor activity of a replication-competent lytic adenovirus through enhanced cytotoxicity and radiosensitization. Hum. Gene Ther., 11: 67-76, 2000.
12. Paielli, D. L., Wing, M., Rogulski, K. R., Gilbert, J. D., Kolozsvary, A., Kim, J. H., Hughes, J. V., Schnell, M., Thompson, T., and Freytag S. O. Evaluation of the biodistribution, toxicity, and potential of germ line transmission of a replication-competent human adenovirus following intraprostatic administration in the mouse. Molecular Ther. 1: 263-274, 2000.
13. Freytag, S. O., Paielli, D., Wing, M., Rogulski, K., Brown, S., Kolozsvary, A., Seely, J., Barton, K., Dragovic, A., and Kim, J. H. Efficacy and toxicity of replication-competent adenovirus-mediated double suicide gene therapy in combination with radiation therapy in an orthotopic mouse prostate cancer model. Int. J. Radiat. Oncol. Biol. Phys., 54: 873-886, 2002.
14. Freytag, S. O., Khil, M., Stricker, H., Peabody, J., Menon, M., DePeralta-Venturina, M., Nafziger, D., Pegg, J., Paielli, D., Brown, S., Barton, K., Lu, M., Aguilar-Cordova, E., and Kim, J. H. Phase I study of replication-competent adenovirus-mediated double suicide gene therapy for the treatment of locally recurrent prostate cancer. Cancer Res., 62: 4968-4976, 2002.
15. Freytag, S. O., Stricker, H., Peabody, J., Menon, M., DePeralta-Venturina, M., Pegg, J., Paiellii, D., Brown, S., Lu, M., and Kim, J. H. Phase I study of replication-competent-adenovirus-mediated double suicide gene therapy in combination with conventional dose three-dimensional conformal radiation therapy for the treatment of locally aggressive prostate cancer. In preparation, 2003.
16. Black, M., Kokoris, M., and Sabo, P. Herpes simplex virus-1 thymidine kinase mutants created by semi-random sequence mutagenesis improve prodrug-mediated tumor cell killing. Cancer Res., 61: 3022-3026, 2001.
17. Kievit, E., Bershad, E., Ng, E., Sethna, P., Dev, I., Lawrence, T., Rehemtulla, A. Superiority of yeast over bactedal cytosine deaminase for enzyme/prodrug gene therapy in colon cancer xenografts. Cancer Res., 59: 1417-1421, 1999.
18. Bischoff J R, Kirn D H, Williams A, Heise C, Horn S, Muna M, et al. An adenovirus mutant that replicates selectively in p53-deficient human tumor cells. Science, 274: 373-376, 1996.
19. Heise C, Sampson-Johannes A, Williams A, McCormick F, von Hoff D D, Kirn D H. ONYX-015, an E1B gene-attenuated adenovirus, causes tumor-specific cytolysis and antitumoral efficacy that can be augmented by standard chemotherapeutic agents. Nature Med. 3: 639-645, 1997.
20. Ganly, I., Kirn, D., Eckhardt, S., Rodriguez, G., Soutar, D., Otto, R., Robertson, A., Park, O., Gulley, M., Heise, C., von Hoff, D., and Kaye, S. A phase I study of ONYX-015, an E1B attenuated adenovirus, administered intratumorally to patents with recurrent head and neck cancer. Clin. Cancer Res., 6: 798-806, 2000.
21. Nemunaitis, J., Khuri, F., Ganly, I., Arseneau, J., Posner, M., Vokes, E., Kuhn, J., McCarty, T., Landers, S., Blackburn, A., Romel, L., Randlev, B., Kaye, S., and Kim, D. Phase II trial of intratumoral administration of ONYX-015, a replication-selective adenovirus, in patients with refractory head and neck cancer. J. Clin. Oncol., 19: 289-298, 2001.
22. Nemunaitis, J., Ganly, I., Khuri, F., Arsenead, J, Kuhn, J., McCarty, T., Landers, S., Maples, P., Romel, L., Randlev, B., Reid, T., Kaye, S., and Kim, D. Selective replication and oncolysis in p53 mutant tumors with ONYX-015, an E1B-55 kD gene-deleted adenovirus, in patients with advanced head and neck cancer: a phase II trial. Cancer Res., 60: 6359-6366, 2000.
23. Khuri, F., Nemunaitis, J., Ganly, I., Arseneau, J., Tannock, I., Romel., L., Gore, M., Ironside, J., MacDougall, R., Heise, C., Randlev, B., Gillenwater, A., Bruso, P., Kaye, S., Hong, W., and Kirn, D. A controlled trial of intratumoral ONYX-015, a selectively-replicating adenovirus, in combination with cisplatin and 5-fluorouracil in patients with recurrent head and neck cancer. Nature Med., 6: 879-885, 2000.
24. Mulvihill, S,, Warren, R., Venook, A., Adler, A., Randlev, B., Heise, C., and Kirn, D. Safety and feasibility of injection with an E1B-55 kDa gene-deleted, replication-selective adenovirus (ONYX-015) into primary carcinomas of the pancreas: a phase I trial. Gene Ther., 8: 308-315, 2001.
25. Reid, T., Galanis, E., Abbruzzese, J., Sze, D., Andrews, J., Romel, L., Hatfield, M., Rubin, J., and Kim, D. Intra-arterial administration of a replication-selective adenovirus (dl1520) in patients with colorectal carcinoma metastatic to the liver: a phase I trial. Gene Ther., 8: 1618-1626, 2001.
26. Vasey, P., Shulman, L., Campos, S., Davis, J., Gore, M., Johnston, S., Kirn, D., O'Neill, V., Siddiqui, N., Seiden, M., and Kaye, S. Phase I trial of intraperitoneal injection of the E1B-55-kd-gene-deleted adenovirus ONYX-015 (dl520) given on days 1 through 5 every 3 weeks in patients with recurrent/refractory epithelial ovarian cancer. J. Clin. Oncol, 20: 1562-1569, 2002.
27. Rodriquez, R., Schuur, E., Lim, H., Henderson, G., Simons, J., and Henderson, D. Prostate attenuated replication competent adenovirus (ARCA) CN706: a selective cytotoxic for prostate-specific antigen-positive prostate cancer cells. Cancer Res., 57: 2559-2563, 1997.
28. Chen, Y., DeWeese, T., Dilley, J., Zhang, Y., Li, Y., Ramesh, N., Lee, J., Pennathur-Das, R., Radzyminski, J., Wypych, J., Brignetti, D., Scott, S., Stephens, J., Karpf, D., Henderson, D. and Yu, D. CV706, a prostate cancer-specific adenovirus variant, in combination with radiotherapy produces synergistic antitumor efficacy without increasing toxicity. Cancer Res., 61: 5453-5460, 2001.
29. DeWeese, T., van der Poel, H., Li, S., Mikhak, B., Drew, R., Goemann, M., Hamper, U., DeJong, R., Detorie, N., Roddguez, R., Haulk, T., DeMarzo, A., Piantadosi, S., Yu, D., Chen, Y., Henderson, D., Carducci, M., Nelson, W., and Simons, J. A phase I trial of CV706, a replication-competent, PSA selective oncolytic adenovirus, for the treatment of locally recurrent prostate cancer following radiation therapy. Cancer Res., 61: 7464-7472, 2001.
30. Burke and Olson, “Preparation of Clone Libraries in Yeast Artificial-Chromosome Vectors” in Methods in Enzymology, Vol. 194, “Guide to Yeast Genetics and Molecular Biology”, eds. C. Guthrie and G. Fink, Academic Press, Inc., Chap. 17, pp. 251-270 (1991).
31. Capecchi, “Altering the genome by homologous recombination” Science 244:1288-1292 (1989).
32. Davies et al., “Targeted alterations in yeast artificial chromosomes for inter-species gene transfer”, Nucleic Acids Research, Vol. 20, No. 11, pp. 2693-2698 (1992).
33. Dickinson et al., “High frequency gene targeting using insertional vectors”, Human Molecular Genetics, Vol. 2, No. 8, pp. 1299-1202 (1993).
34. Duff and Lincoln, “Insertion of a pathogenic mutation into a yeast artificial chromosome containing the human APP gene and expression in ES cells”, Research Advances in Alzheimers Disease and Related Disorders, 1995.
35. Huxley et al., “The human, HPRT gene on a yeast artificial chromosome is functional when transferred to mouse cells by cell fusion”, Genomics, 9:742-750 (1991).
36. Jakobovits et al., “Germ-line transmission and expression of a human-derived yeast artificial chromosome”, Nature, Vol. 362, pp. 255-261 (1993).
37. Lamb et al., “Introduction and expression of the 400 kilobase precursor amyloid protein gene in transgenic mice”, Nature Genetics, Vol. 5, pp. 22-29 (1993).
38. Pearson and Choi, Expression of the human b-amyloid precursor protein gene from a yeast artificial chromosome in transgenic mice. Proc. Natl. Acad. Sci. USA, 1993. 90, 10578-82.
39. Rothstein, “Targeting, disruption, replacement, and allele rescue: integrative DNA transformation in yeast” in Methods in Enzymology, Vol. 194, “Guide to Yeast Genetics and Molecular Biology”, eds. C., Guthrie and G. Fink, Academic Press, Inc., Chap. 19, pp. 281-301 (1991).
40. Schedl et al., “A yeast artificial chromosome covering the tyrosinase gene confers copy number-dependent expression in transgenic mice”, Nature, Vol. 362, pp. 258-261 (1993).
41. Strauss et al., “Germ line transmission of a yeast artificial chromosome spanning the murine a (1) collagen locus”, Science, Vol. 259, pp. 1904-1907 (1993).
42. Gilboa, E, Eglitis, M A, Kantoff, P W, Anderson, W F: Transfer and expression of cloned genes using retroviral vectors. BioTechniques 4(6):504-512, 1986.
43. Cregg J M, Vedvick T S, Raschke W C: Recent Advances in the Expression of Foreign Genes in Pichia pastoris, Bio/Technology 11:905-910, 1993.
44. Culver, 1998. Site-Directed recombination for repair of mutations in the human ADA gene. (Abstract) Antisense DNA & RNA based therapeutics, February, 1998, Coronado, Calif.
45. Huston et al, 1991 “Protein engineering of single-chain Fv analogs and fusion proteins” in Methods in Enzymology (J J Langone, ed.; Academic Press, New York, N.Y.) 203:46-88.
46. Johnson and Bird, 1991 “Construction of single-chain Fvb derivatives of monoclonal antibodies and their production in Escherichia coli in Methods in Enzymology (J J Langone, ed.; Academic Press, New York, N.Y.) 203:88-99.
47. Mernaugh and Mernaugh, 1995 “An overview of phage-displayed recombinant antibodies” in Molecular Methods In Plant Pathology (R P Singh and U S Singh, eds.; CRC Press Inc., Boca Raton, Fla.) pp. 359-365.
47. Mernaugh and Mernaugh, 1995 “An overview of phage-displayed recombinant antibodies” in Molecular Methods In Plant Pathology (RP Singh and U S Singh, eds.; CRC Press Inc., Boca Raton, Fla.) pp. 359-365.

Complete DNA Sequence of Ad5-yCD/mutTK_{SR39}rep-ADP Virus

(SEQ. ID NO. 1)

TCCCTTCCAGCTCTCTGCCCCTTTTGGATTGAAGCCAATATGATAATGAGGGGGTGGAGTTTGTGACGTGGCGCGGGGCGTGGGAACGGGGCGGGT
GACGTAGTAGTGTGGCGGAAGTGTGATGTTGCAAGTGTGGCGGAACACATGTAAGCGACGGATGTGGCAAAAGTGACGTTTTTGGTGTGCGCCGGT
GTACACAGGAAGTGACAATTTTCGCGCGGTTTTAGGCGGATGTTGTAGTAAATTTGGGCGTAACCGAGTAAGATTTGGCCATTTTCGCGGGAAAAC
TGAATAAGAGGAAGTGAAATCTGAATAATTTTGTGTTACTCATAGCGCGTAATCTCTAGCATCGAAGATCCATTTGTCTAGGGCCGCGGGGACTTT
GACCGTTTACGTGGAGACTCGCCCAGGTGTTTTTCTCAGGTGTTTTCCGCGTTCCGGGTCAAAGTTGGCGTTTTATTATTATAGTCAGCTGACGTG
TAGTGTATTTATACCCGGTGAGTTCCTCAAGAGGCCACTCTTGAGTGCCAGCGAGTAGAGTTTTCTCCTCCGAGCCGCTCCGACACCGGGACTGAA
AATGAGACATATTATCTGCCACGGAGGTGTTATTACCGAAGAAATGGCCGCCAGTCTTTTGGACCAGCTGATCGAAGAGGTACTGGCTGATAATCT
TCCACCTCCTAGCCATTTTGAACCACCTACCCTTCACGAACTGTATGATTTAGACGTGACGGCCCCCGAAGATCCCAACGAGGAGGCGGCTTCGCA
GATTTTTCCCGACTCTGTAATGTTGGCGGTGCAGGAAGGGATTGACTTACTCACTTTTCCGCCGGCGCCCGGTTCTCCGGAGCCGCCTCACCTTTC
CCGGCAGCCCGAGCAGCCGGAGCAGAGAGCCTTGGGTCCGGTTTCTATGCCAAACCTTGTACCGGAGGTGATCGATCTTACCTGCCACGAGGCTGG
CTTTCCACCCAGTGACGACGAGGATGAAGAGGGTGAGGAGTTTGTGTTAGATTATGTGGAGCACCCCGGGCACGGTTGCAGGTCTTGTCATTATCA
CCGGAGGAATACGGGGGACCCAGATATTATGTGTTCGCTTTGCTATATGAGGACCTGTGGCATGTTTGTCTACAGTAAGTGAAAATTATGGGCAGT
GGGTGATAGAGTGGTGGGTTTGGTGTGGTAATTTTTTTTTTAATTTTTACAGTTTTGTGGTTTAAAGAATTTTGTATTGTGATTTTTTTAAAAGGT
CCTGTGTCTGAACCTGAGCCTGAGCCCGAGCCAGAACCGGAGCCTGCAAGACCTACCCGCCGTCCTAAAATGGCGCCTGCTATCCTGAGACGCCCG
ACATCACCTGTGTCTAGAGAATGCAATAGTAGTACGGATAGCTGTGACTCCGGTCCTTCTAACACACCTCCTGAGATACACCCGGTGGTCCCGCTG
TGCCCCATTAAACCAGTTGCCGTGAGAGTTGGTGGGCGTCGCCAGGCTGTGGAATGTATCGAGGACTTGCTTAACGAGCCTGGGCAACCTTTGGAC
TTGAGCTGTAAACGCCCCAGGCCATAAGGTGTAAACCTGTGATTGCGTGTGTGGTTAACGCCTTTGTTTGCTGAATGAGTTGATGTAAGTTTAATA
AAGGGTGAGATAATGTTTAACTTGCATGGCGTGTTAAATGGGGCGGGGCTTAAAGGGTATATAATGCGCCGTGGGCTAATCTTGGTTACATCTGAC
CTCATGGAGGCTTGGGAGTGTTTGGAAGATTTTTCTGCTGTGCGTAACTTGCTGGAACAGAGCTCTAACAGTACCTCTTGGTTTTGGAGGTTTCTG
TGGGGCTCATCCCAGGCAAAGTTAGTCTGCAGAATTAAGGAGGATTACAAGTGGGAATTTGAAGAGCTTTTGAAATCCTGTGGTGAGCTGTTTGAT
TCTTTGAATCTGGGTCACCAGGCGCTTTTCCAAGAGAAGGTCATCAAGACTTTGGATTTTTCCACACCGGGGCGCGCTGCGGCTGCTGTTGCTTTT
TTGAGTTTTATAAAGGATAAATGGAGCGAAGAAACCCATCTGAGCGGGGGGTACCTGCTGGATTTTCTGGCCATGCATCTGTGGAGAGCGGTTGTG
AGACACAAGAATCGCCTGCTACTGTTGTCTTCCGTCCGCCCGGCGATAATACCGACGGAGGAGCAGCAGCAGCAGCAGGAGGAAGCCAGGCGGCGG
CGGCAGGAGCAGAGCCCATGGAACCCGAGAGCCGGCCTGGACCCTCGGGAATGAATGTTGTATAGGTGGCTTAACTGTATAGATCTAATTCCCTGG
CATTATGCCCAGTACATGACCTTATGGGACTTTCCTACTTGGCAGTACATCTACGTATTAGTCATCGCTATTACCATGGTGATGCGGTTTTGGCAG
TACATCAATGGGCGTGGATAGCGGTTTGACTCACGGGGATTTCCAAGTCGCCACCCCATTGACGTCAATGGGAGTTTGTTTTGGCACCAAAATCAA
CGGGACTTTCCAAAATGTCGTAACAACTCCGCCCCATTGACGCAAATGGGCGGTAGGCGTGTACGGTGGGAGGTCTATATAAGCAGAGCTCGTTTA
GTGAACCGTCAGATCGCCTGGAGACGCCATCCACGCTGTTTTGACCTCCATAGAAGACACCGGGACCGATCCAGCCTGGGGATCTTCGAGTCGAGG
GATCCCTCGAGCCACCATGGGGACAGGGGGAATGGCAAGCAAGTGGGATCAGAAGGGTATGGACATTGCCTATGAGGAGGCGGCCTTAGGTTACAA
AGAGGGTGGTGTTCCTATTGGCGGATGTCTTATCAATAACAAAGACGGAAGTGTTCTCGGTCGTGGTCACAACATGAGATTTCAAAAGGGATCCGC
CACACTACATGGTGAGATCTCCACTTTGGAAAACTGTGGGAGATTAGAGGGCAAAGTGTACAAAGATACCACTTTGTATACGACGCTGTCTCCATG
CGACATGTGTACAGGTGCCATCATCATGTATGGTATTCCACGCTGTGTTGTCGGTGAGAACGTTAATTTCAAAAGTAAGGGCGAGAAATATTTACA
AACTAGAGGTCACGAGGTTGTTGTTGTTGACGATGAGAGGTGTAAAAAGATCATGAAACAATTTATCGATGAAAGACCTCAGGATTGGTTTGAAGA
TATTGGTGAGGGTGGAGGGGGAGGCGGTGGGGGAGGTGCTAGCATGGCTTCGTACCCCTGCCATCAACACGCGTCTGCGTTCGACCAGGCTGCGCG
TTCTCGCGGCCATAGCAACCGACGTACGGCGTTGCGCCCTCGCCGGCAGCAAGAAGCCACGGAAGTCCGCCTGGAGCAGAAAATGCCCACGCTACT
GCGGGTTTATATAGACGGTCCTCACGGGATGGGGAAAACCACCACCACGCAACTGCTGGTGGCCCTGGGTTCGCGCGACGATATCGTCTACGTACC
CGAGCCGATGACTTACTGGCAGGTGCTGGGGGCTTCCGAGACAATCGCGAACATCTACACCACACAACACCGCCTCGACCAGGGTGAGATATCGGC
CGGGGACGCGGCGGTGGTAATGACAAGCGCCCAGATAACAATGGGCATGCCTTATGCCGTGACCGACGCCGTTCTGGCTCCTCATGTCGGGGGGGA
GGCTGGGAGTTCACATGCCCCGCCCCCGGCCCTCACCATCTTCCTCGACCGCCATCCCATCGCCTTCATGCTGTGCTACCCGGCCGCGCGGTACCT
TATGGGCAGCATGACCCCCCAGGCCGTGCTGGCGTTCGTGGCCCTCATCCCGCCGACCTTGCCCGGCACAAACATCGTGTTGGGGGCCCTTCCGGA
GGACAGACACATCGACCGCCTGGCCAAACGCCAGCGCCCCGGCGAGCGGCTTGACCTGGCTATGCTGGCCGCGATTCGCCGCGTTTACGGGCTGCT
TGCCAATACGGTGCGGTATCTGCAGGGCGGCGGGTCGTGGTGGGAGGATTGGGGACAGCTTTCGGGGACGGCCGTGCCGCCCCAGGGTGCCGAGCC
CCAGAGCAACGCGGGCCCACGACCCCATATCGGGGACACGTTATTTACCCTGTTTCGGGCCCCCGAGTTGCTGGCCCCCAACGGCGACCTGTATAA
CGTGTTTGCCTGGGCCTTGGACGTCTTGGCCAAACGCCTCCGTCCCATGCACGTCTTTATCCTGGATTACGACCAATCGCCCGCCGGCTGCCGGGA
CGCCCTGCTGCAACTTACCTCCGGGATGGTCCAGACCCACGTCACCACCCCAGGCTCCATACCGACGATCTGCGACCTGGCGCGCACGTTTGCCCG
GGAGATGGGGGAGGCTAACTGAAACACGGAAGAATTCAAGCTTGTCGACTTCGAGCAACTTGTTTATTGCAGCTTATAATGGTTACAAATAAAGCA
ATAGCATCACAAATTTCACAAATAAAGCATTTTTTTCACTGCATTCTAGTTGTGGTTTGTCCAAACTCATCAATGTATCTTATCATGTCTGGATCG
TCTAGCATCGAAGATCTGGATCTGGGCGTGGTTAAGGGTGGGAAAGAATATATAAGGTGGGGGTCTTATGTAGTTTTGTATCTGTTTTGCAGCAGC
CGCCGCCGCCATGAGCACCAACTCGTTTGATGGAAGCATTGTGAGCTCATATTTGACAACGCGCATGCCCCCATGGGCCGGGGTGCGTCAGAATGT
GATGGGCTCCAGCATTGATGGTCGCCCCGTCCTGCCCGCAAACTCTACTACCTTGACCTACGAGACCGTGTCTGGAACGCCGTTGGAGACTGCAGC
CTCCGCCGCCGCTTCAGCCGCTGCAGCCACCGCCCGCGGGATTGTGACTGACTTTGCTTTCCTGAGCCCGCTTGCAAGCAGTGCAGCTTCCCGTTC
ATCCGCCCGCGATGACAAGTTGACGGCTCTTTTGGCACAATTGGATTCTTTGACCCGGGAACTTAATGTCGTTTCTCAGCAGCTGTTGGATCTGCG
CCAGCAGGTTTCTGCCCTGAAGGCTTCCTCCCCTCCCAATGCGGTTTAAAACATAAATAAAAAACCAGACTCTGTTTGGATTTGGATCAAGCAAGT
GTCTTGCTGTCTTTATTTAGGGGTTTTGCGCGCGCGGTAGGCCCGGGACCAGCGGTCTCGGTCGTTGAGGGTCCTGTGTATTTTTTCCAGGACGTG
GTAAAGGTGACTCTGGATGTTCAGATACATGGGCATAAGCCCGTCTCTGGGGTGGAGGTAGCACCACTGCAGAGCTTCATGCTGCGGGGTGGTGTT
GTAGATGATCCAGTCGTAGCAGGAGCGCTGGGCGTGGTGCCTAAAAATGTCTTTCAGTAGCAAGCTGATTGCCAGGGGCAGGCCCTTGGTGTAAGT
GTTTACAAAGCGGTTAAGCTGGGATGGGTGCATACGTGGGGATATGAGATGCATCTTGGACTGTATTTTTAGGTTGGCTATGTTCCCAGCCATATC
CCTCCGGGGATTCATGTTGTGCAGAACCACCAGCACAGTGTATCCGGTGCACTTGGGAAATTTGTCATGTAGCTTAGAAGGAAATGCGTGGAAGAA
CTTGGAGACGCCCTTGTGACCTCCAAGATTTTCCATGCATTCGTCCATAATGATGGCAATGGGCCCACGGGCGGCGGCCTGGGCGAAGATATTTCT
GGGATCACTAACGTCATAGTTGTGTTCCAGGATGAGATCGTCATAGGCCATTTTTACAAAGCGCGGGCGGAGGGTGCCAGACTGCGGTATAATGGT
TCCATCCGGCCCAGGGGCGTAGTTACCCTCACAGATTTGCATTTCCCACGCTTTGAGTTCAGATGGGGGGATCATGTCTACCTGCGGGGCGATGAA
GAAAACGGTTTCCGGGGTAGGGGAGATCAGCTGGGAAGAAAGCAGGTTCCTGAGCAGCTGCGACTTACCGCAGCCGGTGGGCCCGTAAATCACACC
TATTACCGGGTGCAACTGGTAGTTAAGAGAGCTGCAGCTGCCGTCATCCCTGAGCAGGGGGGCCACTTCGTTAAGCATGTCCCTGACTCGCATGTT
TTCCCTGACCAAATCCGCCAGAAGGCGCTCGCCGCCCAGCGATAGCAGTTCTTGCAAGGAAGCAAAGTTTTTCAACGGTTTGAGACCGTCCGCCGT
AGGCATGCTTTTGAGCGTTTGACCAAGCAGTTCCAGGCGGTCCCACAGCTCGGTCACCTGCTCTACGGCATCTCGATCCAGCATATCTCCTCGTTT
CGCGGGTTGGGGCGGCTTTCGCTGTACGGCAGTAGTCGGTGCTCGTCCAGACGGGCCAGGGTCATGTCTTTCCACGGGCGCAGGGTCCTCGTCAGC
GTAGTCTGGGTCACGGTGAAGGGGTGCGCTCCGGGCTGCGCGCTGGCCAGGGTGCGCTTGAGGCTGGTCCTGCTGGTGCTGAAGCGCTGCCGGTCT
TCGCCCTGCGCGTCGGCCAGGTAGCATTTGACCATGGTGTCATAGTCCAGCCCCTCCGCGGCGTGGCCCTTGGCGCGCAGCTTGCCCTTGGAGGAG
GCGCCGCACGAGGGGCAGTGCAGACTTTTGAGGGCGTAGAGCTTGGGCGCGAGAAATACCGATTCCGGGGAGTAGGCATCCGCGCCGCAGGCCCCG
CAGACGGTCTCGCATTCCACGAGCCAGGTGAGCTCTGGCCGTTCGGGGTCAAAAACCAGGTTTCCCCCATGCTTTTTGATGCGTTTCTTACCTCTG
GTTTCCATGAGCCGGTGTCCACGCTCGGTGACGAAAAGGCTGTCCGTGTCCCCGTATACAGACTTGAGAGGCCTGTCCTCGAGCGGTGTTCCGCGG
TCCTCCTCGTATAGAAACTCGGACCACTCTGAGACAAAGGCTCGCGTCCAGGCCAGCACGAAGGAGGCTAAGTGGGAGGGGTAGCGGTCGTTGTCC
ACTAGGGGGTCCACTCGCTCCAGGGTGTGAAGACACATGTCGCCCTCTTCGGCATCAAGGAAGGTGATTGGTTTGTAGGTGTAGGCCACGTGACCG
GGTGTTCCTGAAGGGGGGCTATAAAAGGGGGTGGGGGCGCGTTCGTCCTCACTCTCTTCCGCATCGCTGTCTGCGAGGGCCAGCTGTTGGGGTGAG
TACTCCCTCTGAAAAGCGGGCATGACTTCTGCGCTAAGATTGTCAGTTTCCAAAAACGAGGAGGATTTGATATTCACCTGGCCCGCGGTGATGCCT
TTGAGGGTGGCCGCATCCATCTGGTCAGAAAAGACAATCTTTTTGTTGTCAAGCTTGGTGGCAAACGACCCGTAGAGGGCGTTGGACAGCAACTTG
GCGATGGAGCGCAGGGTTTGGTTTTTGTCGCGATCGGCGCGCTCCTTGGCCGCGATGTTTAGCTGCACGTATTCGCGCGCAACGCACCGCCATTCG
GGAAAGACGGTGGTGCGCTCGTCGGGCACCAGGTGCACGCGCCAACCGCGGTTGTGCAGGGTGACAAGGTCAACGCTGGTGGCTACCTCTCCGCGT
AGGCGCTCGTTGGTCCAGCAGAGGCGGCCGCCCTTGCGCGAGCAGAATGGCGGTAGGGGGTCTAGCTGCGTCTCGTCCGGGGGGTCTGCGTCCACG
GTAAAGACCCCGGGCAGCAGGCGCGCGTCGAAGTAGTCTATCTTGCATCCTTGCAAGTCTAGCGCCTGCTGCCATGCGCGGGCGGCAAGCGCGCGC
TCGTATGGGTTGAGTGGGGGACCCCATGGCATGGGGTGGGTGAGCGCGGAGGCGTACATGCCGCAAATGTCGTAAACGTAGAGGGGCTCTCTGAGT
ATTCCAAGATATGTAGGGTAGCATCTTCCACCGCGGATGCTGGCGCGCACGTAATCGTATAGTTCGTGCGAGGGAGCGAGGAGGTCGGGACCGAGG
TTGCTACGGGCGGGCTGCTCTGCTCGGAAGACTATCTGCCTGAAGATGGCATGTGAGTTGGATGATATGGTTGGACGCTGGAAGACGTTGAAGCTG
GCGTCTGTGAGACCTACCGCGTCACGCACGAAGGAGGCGTAGGAGTCGCGCAGCTTGTTGACCAGCTCGGCGGTGACCTGCACGTCTAGGGCGCAG
TAGTCCAGGGTTTCCTTGATGATGTCATACTTATCCTGTCCCTTTTTTTTCCACAGCTCGCGGTTGAGGACAAACTCTTCGCGGTCTTTCCAGTAC
TCTTGGATCGGAAACCCGTCGGCCTCCGAACGGTAAGAGCCTAGCATGTAGAACTGGTTGACGGCCTGGTAGGCGCAGCATCCCTTTTCTACGGGT
AGCGCGTATGCCTGCGCGGCCTTCCGGAGCGAGGTGTGGGTGAGCGCAAAGGTGTCCCTGACCATGACTTTGAGGTACTGGTATTTGAAGTCAGTG
TCGTCGCATCCGCCCTGCTCCCAGAGCAAAAAGTCCGTGCGCTTTTTGGAACGCGGATTTGGCAGGGCGAAGGTGACATCGTTGAAGAGTATCTTT
CCCGCGCGAGGCATAAAGTTGCGTGTGATGCGGAAGGGTCCCGGCACCTCGGAACGGTTGTTAATTACCTGGGCGGCGAGCACGATCTCGTCAAAG
CCGTTGATGTTGTGGCCCACAATGTAAAGTTCCAAGAAGCGCGGGATGCCCTTGATGGAAGGCAATTTTTTAAGTTCCTCGTAGGTGAGCTCTTCA
GGGGAGCTGAGCCCGTGCTCTGAAAGGGCCCAGTCTGCAAGATGAGGGTTGGAAGCGACGAATGAGCTCCACAGGTCACGGGCCATTAGCATTTGC
AGGTGGTCGCGAAAGGTCCTAAACTGGCGACCTATGGCCATTTTTTCTGGGGTGATGCAGTAGAAGGTAAGCGGGTCTTGTTCCCAGCGGTCCCAT
CCAAGGTTCGCGGCTAGGTCTCGCGCGGCAGTCACTAGAGGCTCATCTCCGCCGAACTTCATGACCAGCATGAAGGGCACGAGCTGCTTCCCAAAG
GCCCCCATCCAAGTATAGGTCTCTACATCGTAGGTGACAAAGAGACGCTCGGTGCGAGGATGCGAGCCGATCGGGAAGAACTGGATCTCCCGCCAC
CAATTGGAGGAGTGGCTATTGATGTGGTGAAAGTAGAAGTCCCTGCGACGGGCCGAACACTCGTGCTGGCTTTTGTAAAAACGTGCGCAGTACTGG
CAGCGGTGCACGGGCTGTACATCCTGCACGAGGTTGACCTGACGACCGCGCACAAGGAAGCAGAGTGGGAATTTGAGCCCCTCGCCTGGCGGGTTT
GGCTGGTGGTCTTCTACTTCGGCTGCTTGTCCTTGACCGTCTGGCTGCTCGAGGGGAGTTACGGTGGATCGGACCACCACGCCGCGCGAGCCCAAA
GTCCAGATGTCCGCGCGCGGCGGTCGGAGCTTGATGACAACATCGCGCAGATGGGAGCTGTCCATGGTCTGGAGCTCCCGCGGCGTCAGGTCAGGC
GGGAGCTCCTGCAGGTTTACCTCGCATAGACGGGTCAGGGCGCGGGCTAGATCCAGGTGATACCTAATTTCCAGGGGCTGGTTGGTGGCGGCGTCG
ATGGCTTGCAAGAGGCCGCATCCCCGCGGCGCGACTACGGTACCGCGCGGCGGGCGGTGGGCCGCGGGGGTGTCCTTGGATGATGCATCTAAAAGC
GGTGACGCGGGCGAGCCCCCGGAGGTAGGGGGGGCTCCGGACCCGCCGGGAGAGGGGGCAGGGGCACGTCGGCGCCGCGCGCGGGCAGGAGCTGGT
GCTGCGCGCGTAGGTTGCTGGCGAACGCGACGACGCGGCGGTTGATCTCCTGAATCTGGCGCCTCTGCGTGAAGACGACGGGCCCGGTGAGCTTGA
GCCTGAAAGAGAGTTCGACAGAATCAATTTCGGTGTCGTTGACGGCGGCCTGGCGCAAAATCTCCTGCACGTCTCCTGAGTTGTCTTGATAGGCGA
TCTCGGCCATGAACTGCTCGATCTCTTCCTCCTGGAGATCTCCGCGTCCGGCTCGCTCCACGGTGGCGGCGAGGTCGTTGGAAATGCGGGCCATGA
GCTGCGAGAAGGCGTTGAGGCCTCCCTCGTTCCAGACGCGGCTGTAGACCACGCCCCCTTCGGCATCGCGGGCGCGCATGACCACCTGCGCGAGAT
TGAGCTCCACGTGCCGGGCGAAGACGGCGTAGTTTCGCAGGCGCTGAAAGAGGTAGTTGAGGGTGGTGGCGGTGTGTTCTGCCACGAAGAAGTACA
TAACCCAGCGTCGCAACGTGGATTCGTTGATATCCCCCAAGGCCTCAAGGCGCTCCATGGCCTCGTAGAAGTCCACGGCGAAGTTGAAAAACTGGG
AGTTGCGCGCCGACACGGTTAACTCCTCCTCCAGAAGACGGATGAGCTCGGCGACAGTGTCGCGCACCTCGCGCTCAAAGGCTACAGGGGCCTCTT
CTTCTTCTTCAATCTCCTCTTCCATAAGGGCCTCCCCTTCTTCTTCTTCTGGCGGCGGTGGGGGAGGGGGGACACGGCGGCGACGACGGCGCACCG
GGAGGCGGTCGACAAAGCGCTCGATCATCTCCCCGCGGCGACGGCGCATGGTCTCGGTGACGGCGCGGCCGTTCTCGCGGGGGCGCAGTTGGAAGA
CGCCGCCCGTCATGTCCCGGTTATGGGTTGGCGGGGGGCTGCCATGCGGCAGGGATACGGCGCTAACGATGCATCTCAACAATTGTTGTGTAGGTA
CTCCGCCGCCGAGGGACCTGAGCGAGTCCGCATCGACCGGATCGGAAAACCTCTCGAGAAAGGCGTCTAACCAGTCACAGTCGCAAGGTAGGCTGA
GCACCGTGGCGGGCGGCAGCGGGCGGCGGTCGGGGTTGTTTCTGGCGGAGGTGCTGCTGATGATGTAATTAAAGTAGGCGGTCTTGAGACGGCGGA
TGGTCGACAGAAGCACCATGTCCTTGGGTCCGGCCTGCTGAATGCGCAGGCGGTCGGCCATGCCCCAGGCTTCGTTTTGACATCGGCGCAGGTCTT
TGTAGTAGTCTTGCATGAGCCTTTCTACCGGCACTTCTTCTTCTCCTTCCTCTTGTCCTGCATCTCTTGCATCTATCGCTGCGGCGGCGGCGGAGT
TTGGCCGTAGGTGGCGCCCTCTTCCTCCCATGCGTGTGACCCCGAAGCCCCTCATCGGCTGAAGCAGGGCTAGGTCGGCGACAACGCGCTCGGCTA
ATATGGCCTGCTGCACCTGCGTGAGGGTAGACTGGAAGTCATCCATGTCCACAAAGCGGTGGTATGCGCCCGTGTTGATGGTGTAAGTGCAGTTGG
CCATAACGGACCAGTTAACGGTCTGGTGACCCGGCTGCGAGAGCTCGGTGTACCTGAGACGCGAGTAAGCCCTCGAGTCAAATACGTAGTCGTTGC
AAGTCCGCACCAGGTACTGGTATCCCACCAAAAAGTGCGGCGGCGGCTGGCGGTAGAGGGGCCAGCGTAGGGTGGCCGGGGCTCCGGGGGCGAGAT
CTTCCAACATAAGGCGATGATATCCGTAGATGTACCTGGACATCCAGGTGATGCCGGCGGCGGTGGTGGAGGCGCGCGGAAAGTCGCGGACGCGGT
TCCAGATGTTGCGCAGCGGCAAAAAGTGCTCCATGGTCGGGACGCTCTGGCCGGTCAGGCGCGCGCAATCGTTGACGCTCTACCGTGCAAAAGGAG
AGCCTGTAAGCGGGCACTCTTCCGTGGTCTGGTGGATAAATTCGCAAGGGTATCATGGCGGACGACCGGGGTTCGAGCCCCGTATCCGGCCGTCCG
CCGTGATCCATGCGGTTACCGCCCGCGTGTCGAACCCAGGTGTGCGACGTCAGACAACGGGGGAGTGCTCCTTTTGGCTTCCTTCCAGGCGCGGCG
GCTGCTGCGCTAGCTTTTTTGGCCACTGGCCGCGCGCAGCGTAAGCGGTTAGGCTGGAAAGCGAAAGCATTAAGTGGCTCGCTCCCTGTAGCCGGA
GGGTTATTTTCCAAGGGTTGAGTCGCGGGACCCCCGGTTCGAGTCTCGGACCGGCCGGACTGCGGCGAACGGGGGTTTGCCTCCCCGTCATGCAAG
ACCCCGCTTGCAAATTCCTCCGGAAACAGGGACGAGCCCCTTTTTTGCTTTTCCCAGATGCATCCGGTGCTGCGGCAGATGCGCCCCCCTCCTCAG
CAGCGGCAAGAGCAAGAGCAGCGGCAGACATGCAGGGCACCCTCCCCTCCTCCTACCGCGTCAGGAGGGGCGACATCCGCGGTTGACGCGGCAGCA
GATGGTGATTACGAACCCCCGCGGCGCCGGGCCCGGCACTACCTGGACTTGGAGGAGGGCGAGGGCCTGGCGCGGCTAGGAGCGCCCTCTCCTGAG
CGGTACCCAAGGGTGCAGCTGAAGCGTGATACGCGTGAGGCGTACGTGCCGCGGCAGAACCTGTTTCGCGACCGCGAGGGAGAGGAGCCCGAGGAG
ATGCGGGATCGAAAGTTCCACGCAGGGCGCGAGCTGCGGCATGGCCTGAATCGCGAGCGGTTGCTGCGCGAGGAGGACTTTGAGCCCGACGCGCGA
ACCGGGATTAGTCCCGCGCGCGCACACGTGGCGGCCGCCGACCTGGTAACCGCATACGAGCAGACGGTGAACCAGGAGATTAACTTTCAAAAAAGC
TTTAACAACCACGTGCGTACGCTTGTGGCGCGCGAGGAGGTGGCTATAGGACTGATGCATCTGTGGGACTTTGTAAGCGCGCTGGAGCAAAACCCA
AATAGCAAGCCGCTCATGGCGCAGCTGTTCCTTATAGTGCAGCACAGCAGGGACAACGAGGCATTCAGGGATGCGCTGCTAAACATAGTAGAGCCC
GAGGGCCGCTGGCTGCTCGATTTGATAAACATCCTGCAGAGCATAGTGGTGCAGGAGCGCAGCTTGAGCCTGGCTGACAAGGTGGCCGCCATCAAC
TATTCCATGCTTAGCCTGGGCAAGTTTTACGCCCGCAAGATATACCATACCCCTTACGTTCCCATAGACAAGGAGGTAAAGATCGAGGGGTTCTAC
ATGCGCATGGCGCTGAAGGTGCTTACCTTGAGCGACGACCTGGGCGTTTATCGCAACGAGCGCATCCACAAGGCCGTGAGCGTGAGCCGGCGGCGC
GAGCTCAGCGACCGCGAGCTGATGCACAGCCTGCAAAGGGCCCTGGCTGGCACGGGCAGCGGCGATAGAGAGGCCGAGTCCTACTTTGACGCGGGC
GCTGACCTGCGCTGGGCCCCAAGCCGACGCGCCCTGGAGGCAGCTGGGGCCGGACCTGGGCTGGCGGTGGCACCCGCGCGCGCTGGCAACGTCGGC
GGCGTGGAGGAATATGACGAGGACGATGAGTACGAGCCAGAGGACGGCGAGTACTAAGCGGTGATGTTTCTGATCAGATGATGCAAGACGCAACGG
ACCCGGCGGTGCGGGCGGCGCTGCAGAGCCAGCCGTCCGGCCTTAACTCCACGGACGACTGGCGCCAGGTCATGGACCGCATCATGTCGCTGACTG
CGCGCAATCCTGACGCGTTCCGGCAGCAGCCGCAGGCCAACCGGCTCTCCGCAATTCTGGAAGCGGTGGTCCCGGCGCGCGCAAACCCCACGCACG
AGAAGGTGCTGGCGATCGTAAACGCGCTGGCCGAAAACAGGGCCATCCGGCCCGACGAGGCCGGCCTGGTCTACGACGCGCTGCTTCAGCGCGTGG
CTCGTTACAACAGCGGCAACGTGCAGACCAACCTGGACCGGCTGGTGGGGGATGTGCGCGAGGCCGTGGCGCAGCGTGAGCGCGCGCAGCAGCAGG
GCAACCTGGGCTCCATGGTTGCACTAAACGCCTTCCTGAGTACACAGCCCGCCAACGTGCCGCGGGGACAGGAGGACTACACCAACTTTGTGAGCG
CACTGCGGCTAATGGTGACTGAGACACCGCAAAGTGAGGTGTACCAGTCTGGGCCAGACTATTTTTTCCAGACCAGTAGACAAGGCCTGCAGACCG
TAAACCTGAGCCAGGCTTTCAAAAACTTGCAGGGGCTGTGGGGGGTGCGGGCTCCCACAGGCGACCGCGCGACCGTGTCTAGCTTGCTGACGCCCA
ACTCGCGCCTGTTGCTGCTGCTAATAGCGCCCTTCACGGACAGTGGCAGCGTGTCCCGGGACACATACCTAGGTCACTTGCTGACACTGTACCGCG
AGGCCATAGGTCAGGCGCATGTGGACGAGCATACTTTCCAGGAGATTACAAGTGTCAGCCGCGCGCTGGGGCAGGAGGACACGGGCAGCCTGGAGG
CAACCCTAAACTACCTGCTGACCAACCGGCGGCAGAAGATCCCCTCGTTGCACAGTTTAAACAGCGAGGAGGAGCGCATTTTGCGCTACGTGCAGC
AGAGCGTGAGCCTTAACCTGATGCGCGACGGGGTAACGCCCAGCGTGGCGCTGGACATGACCGCGCGCAACATGGAACCGGGCATGTATGCCTCAA
ACCGGCCGTTTATCAACCGCCTAATGGACTACTTGCATCGCGCGGCCGCCGTGAACCCCGAGTATTTCACCAATGCCATCTTGAACCCGCACTGGC
TACCGCCCCCTGGTTTCTACACCGGGGGATTCGAGGTGCCCGAGGGTAACGATGGATTCCTCTGGGACGACATAGACGACAGCGTGTTTTCCCCGC
AACCGCAGACCCTGCTAGAGTTGCAACAGCGCGAGCAGGCAGAGGCGGCGCTGCGAAAGGAAAGCTTCCGCAGGCCAAGCAGCTTGTCCGATCTAG
GCGCTGCGGCCCCGCGGTCAGATGCTAGTAGCCCATTTCCAAGCTTGATAGGGTCTCTTACCAGCACTCGCACCACCCGCCCGCGCCTGCTGGGCG
AGGAGGAGTACCTAAACAACTCGCTGCTGCAGCCGCAGCGCGAAAAAAACCTGCCTCCGGCATTTCCCAACAACGGGATAGAGAGCCTAGTGGACA
AGATGAGTAGATGGAAGACGTACGCGCAGGAGCACAGGGACGTGCCAGGCCCGCGCCCGCCCACCCGTCGTCAAAGGCACGACCGTCAGCGGGGTC
TGGTGTGGGAGGACGATGACTCGGCAGACGACAGCAGCGTCCTGGATTTGGGAGGGAGTGGCAACCCGTTTGCGCACCTTCGCCCCAGGCTGGGGA
GAATGTTTTAAAAAAAAAAAAGCATGATGCAAAATAAAAAACTCACCAAGGCCATGGCACCGAGCGTTGGTTTTCTTGTATTCCCCTTAGTATGCG
GCGCGCGGCGATGTATGAGGAAGGTCCTCCTCCCTCCTACGAGAGTGTGGTGAGCGCGGCGCCAGTGGCGGCGGCGCTGGGTTCTCCCTTCGATGC
TCCCCTGGACCCGCCGTTTGTGCCTCCGCGGTACCTGCGGCCTACCGGGGGGAGAAACAGCATCCGTTACTCTGAGTTGGCACCCCTATTCGACAC
CACCCGTGTGTACCTGGTGGACAACAAGTCAACGGATGTGGCATCCCTGAACTACCAGAACGACCACAGCAACTTTCTGACCACGGTCATTCAAAA
CAATGACTACAGCCCGGGGGAGGCAAGCACACAGACCATCAATCTTGACGACCGGTCGCACTGGGGCGGCGACCTGAAAACCATCCTGCATACCAA
CATGCCAAATGTGAACGAGTTCATGTTTACCAATAAGTTTAAGGCGCGGGTGATGGTGTCGCGCTTGCCTACTAAGGACAATCAGGTGGAGCTGAA
ATACGAGTGGGTGGAGTTCACGCTGCCCGAGGGCAACTACTCCGAGACCATGACCATAGACCTTATGAACAACGCGATCGTGGAGCACTACTTGAA
AGTGGGCAGACAGAACGGGGTTCTGGAAAGCGACATCGGGGTAAAGTTTGACACCCGCAACTTCAGACTGGGGTTTGACCCCGTCACTGGTCTTGT
CATGCCTGGGGTATATACAAACGAAGCCTTCCATCCAGACATCATTTTGCTGCCAGGATGCGGGGTGGACTTCACCCACAGCCGCCTGAGCAACTT
GTTGGGCATCCGCAAGCGGCAACCCTTCCAGGAGGGCTTTAGGATCACCTACGATGATCTGGAGGGTGGTAACATTCCCGCACTGTTGGATGTGGA
CGCCTACCAGGCGAGCTTGAAAGATGACACCGAACAGGGCGGGGGTGGCGCAGGCGGCAGCAACAGCAGTGGCAGCGGCGCGGAAGAGAACTCCAA
CGCGGCAGCCGCGGCAATGCAGCCGGTGGAGGACATGAACGATCATGCCATTCGCGGCGACACCTTTGCCACACGGGCTGAGGAGAAGCGCGCTGA
GGCCGAAGCAGCGGCCGAAGCTGCCGCCCCCGCTGCGCAACCCGAGGTCGAGAAGCCTCAGAAGAAACCGGTGATCAAACCCCTGACAGAGGACAG
CAAGAAACGCAGTTACAACCTAATAAGCAATGACAGCACCTTCACCCAGTACCGCAGCTGGTACCTTGCATACAACTACGGCGACCCTCAGACCGG
AATCCGCTCATGGACCCTGCTTTGCACTCCTGACGTAACCTGCGGCTCGGAGCAGGTCTACTGGTCGTTGCCAGACATGATGCAAGACCCCGTGAC
CTTCCGCTCCACGCGCCAGATCAGCAACTTTCCGGTGGTGGGCGCCGAGCTGTTGCCCGTGCACTCCAAGAGCTTCTACAACGACCAGGCCGTCTA
CTCCCAACTCATCCGCCAGTTTACCTCTCTGACCCACGTGTTCAATCGCTTTCCCGAGAACCAGATTTTGGCGCGCCCGCCAGCCCCCACCATCAC
CACCGTCAGTGAAAACGTTCCTGCTCTCACAGATCACGGGACGCTACCGCTGCGCAACAGCATCGGAGGAGTCCAGCGAGTGACCATTACTGACGC
CAGACGCCGCACCTGCCCCTACGTTTACAAGGCCCTGGGCATAGTCTCGCCGCGCGTCCTATCGAGCCGCACTTTTTGAGCAAGCATGTCCATCCT
TATATCGCCCAGCAATAACACAGGCTGGGGCCTGCGCTTCCCAAGCAAGATGTTTGGCGGGGCCAAGAAGCGCTCCGACCAACACCCAGTGCGCGT
GCGCGGGCACTACCGCGCGCCCTGGGGCGCGCACAAACGCGGCCGCACTGGGCGCACCACCGTCGATGACGCCATCGACGCGGTGGTGGAGGAGGC
GCGCAACTACACGCCCACGCCGCCACCAGTGTCCACAGTGGACGCGGCCATTCAGACCGTGGTGCGCGGAGCCCGGCGCTATGCTAAAATGAAGAG
ACGGCGGAGGCGCGTAGCACGTCGCCACCGCCGCCGACCCGGCACTGCCGCCCAACGCGCGGCGGCGGCCCTGCTTAACCGCGCACGTCGCACCGG
CCGACGGGCGGCCATGCGGGCCGCTCGAAGGCTGGCCGCGGGTATTGTCACTGTGCCCCCCAGGTCCAGGCGACGAGCGGCCGCCGCAGCAGCCGC
GGCCATTAGTGCTATGACTCAGGGTCGCAGGGGCAACGTGTATTGGGTGCGCGACTCGGTTAGCGGCCTGCGCGTGCCCGTGCGCACCCGCCCCCC
GCGCAACTAGATTGCAAGAAAAAACTACTTAGACTCGTACTGTTGTATGTATCCAGCGGCGGCGGCGCGCAACGAAGCTATGTCCAAGCGCAAAAT
CAAAGAAGAGATGCTCCAGGTCATCGCGCCGGAGATCTATGGCCCCCCGAAGAAGGAAGAGCAGGATTACAAGCCCCGAAAGCTAAAGCGGGTCAA
AAAGAAAAAGAAAGATGATGATGATGAACTTGACGACGAGGTGGAACTGCTGCACGCTACCGCGCCCAGGCGACGGGTACAGTGGAAAGGTCGACG
CGTAAAACGTGTTTTGCGACCCGGCACCACCGTAGTCTTTACGCCCGGTGAGCGCTCCACCCGCACCTACAAGCGCGTGTATGATGAGGTGTACGG
CGACGAGGACCTGCTTGAGCAGGCCAACGAGCGCCTCGGGGAGTTTGCCTACGGAAAGCGGCATAAGGACATGCTGGCGTTGCCGCTGGACGAGGG
CAACCCAACACCTAGCCTAAAGCCCGTAACACTGCAGCAGGTGCTGCCCGCGCTTGCACCGTCCGAAGAAAAGCGCGGCCTAAAGCGCGAGTCTGG
TGACTTGGCACCCACCGTGCAGCTGATGGTACCCAAGCGCCAGCGACTGGAAGATGTCTTGGAAAAAATGACCGTGGAACCTGGGCTGGAGCCCGA
GGTCCGCGTGCGGCCAATCAAGCAGGTGGCGCCGGGACTGGGCGTGCAGACCGTGGACGTTCAGATACCCACTACCAGTAGCACCAGTATTGCCAC
CGCCACAGAGGGCATGGAGACACAAACGTCCCCGGTTGCCTCAGCGGTGGCGGATGCCGCGGTGCAGGCGGTCGCTGCGGCCGCGTCCAAGACCTC
TACGGAGGTGCAAACGGACCCGTGGATGTTTCGCGTTTCAGCCCCCCGGCGCCCGCGCGGTTCGAGGAAGTACGGCGCCGCCAGCGCGCTACTGCC
CGAATATGCCCTACATCCTTCCATTGCGCCTACCCCCGGCTATCGTGGCTACACCTACCGCCCCAGAAGACGAGCAACTACCCGACGCCGAACCAC
CACTGGAACCCGCCGCCGCCGTCGCCGTCGCCAGCCCGTGCTGGCCCCGATTTCCGTGCGCAGGGTGGCTCGCGAAGGAGGCAGGACCCTGGTGCT
GCCAACAGCGCGCTACCACCCCAGCATCGTTTAAAAGCCGGTCTTTGTGGTTCTTGCAGATATGGCCCTCACCTGCCGCCTCCGTTTCCCGGTGCC
GGGATTCCGAGGAAGAATGCACCGTAGGAGGGGCATGGCCGGCCACGGCCTGACGGGCGGCATGCGTCGTGCGCACCACCGGCGGCGGCGCGCGTC
GCACCGTCGCATGCGCGGCGGTATCCTGCCCCTCCTTATTCCACTGATCGCCGCGGCGATTGGCGCCGTGCCCGGAATTGCATCCGTGGCCTTGCA
GGCGCAGAGACACTGATTAAAAACAAGTTGCATGTGGAAAAATCAAAATAAAAAGTCTGGACTCTCACGCTCGCTTGGTCCTGTAACTATTTTGTA
GAATGGAAGACATCAACTTTGCGTCTCTGGCCCCGCGACACGGCTCGCGCCCGTTCATGGGAAACTGGCAAGATATCGGCACCAGCAATATGAGCG
GTGGCGCCTTCAGCTGGGGCTCGCTGTGGAGCGGCATTAAAAATTTCGGTTCCACCGTTAAGAACTATGGCAGCAAGGCCTGGAACAGCAGCACAG
GCCAGATGCTGAGGGATAAGTTGAAAGAGCAAAATTTCCAACAAAAGGTGGTAGATGGCCTGGCCTCTGGCATTAGCGGGGTGGTGGACCTGGCCA
ACCAGGCAGTGCAAAATAAGATTAACAGTAAGCTTGATCCCCGCCCTCCCGTAGAGGAGCCTCCACCGGCCGTGGAGACAGTGTCTCCAGAGGGGC
GTGGCGAAAAGCGTCCGCGCCCCGACAGGGAAGAAACTCTGGTGACGCAAATAGACGAGCCTCCCTCGTACGAGGAGGCACTAAAGCAAGGCCTGC
CCACCACCCGTCCCATCGCGCCCATGGCTACCGGAGTGCTGGGCCAGCACACACCCGTAACGCTGGACCTGCCTCCCCCCGCCGACACCCAGCAGA
AACCTGTGCTGCCAGGCCCGACCGCCGTTGTTGTAACCCGTCCTAGCCGCGCGTCCCTGCGCCGCGCCGCCAGCGGTCCGCGATCGTTGCGGCCCG
TAGCCAGTGGCAACTGGCAAAGCACACTGAACAGCATCGTGGGTCTGGGGGTGCAATCCCTGAAGCGCCGACGATGCTTCTGAATAGCTAACGTGT
CGTATGTGTGTCATGTATGCGTCCATGTCGCCGCCAGAGGAGCTGCTGAGCCGCCGCGCGCCCGCTTTCCAAGATGGCTACCCCTTCGATGATGCC
GCAGTGGTCTTACATGCACATCTCGGGCCAGGACGCCTCGGAGTACCTGAGCCCCGGGCTGGTGCAGTTTGCCCGCGCCACCGAGACGTACTTCAG
CCTGAATAACAAGTTTAGAAACCCCACGGTGGCGCCTACGCACGACGTGACCACAGACCGGTCCCAGCGTTTGACGCTGCGGTTCATCCCTGTGGA
CCGTGAGGATACTGCGTACTCGTACAAGGCGCGGTTCACCCTAGCTGTGGGTGATAACCGTGTGCTGGACATGGCTTCCACGTACTTTGACATCCG
CGGCGTGCTGGACAGGGGCCCTACTTTTAAGCCCTACTCTGGCACTGCCTACAACGCCCTGGCTCCCAAGGGTGCCCCAAATCCTTGCGAATGGGA
TGAAGCTGCTACTGCTCTTGAAATAAACCTAGAAGAAGAGGACGATGACAACGAAGACGAAGTAGACGAGCAAGCTGAGCAGCAAAAAACTCACGT
ATTTGGGCAGGCGCCTTATTCTGGTATAAATATTACAAAGGAGGGTATTCAAATAGGTGTCGAAGGTCAAACACCTAAATATGCCGATAAAACATT
TCAACCTGAACCTCAAATAGGAGAATCTCAGTGGTACGAAACTGAAATTAATCATGCAGCTGGGAGAGTCCTTAAAAAGACTACCCCAATGAAACC
ATGTTACGGTTCATATGCAAAACCCACAAATGAAAATGGAGGGCAAGGCATTCTTGTAAAGCAACAAAATGGAAAGCTAGAAAGTCAAGTGGAAAT
GCAATTTTTCTCAACTACTGAGGCGACCGCAGGCAATGGTGATAACTTGACTCCTAAAGTGGTATTGTACAGTGAAGATGTAGATATAGAAACCCC
AGACACTCATATTTCTTACATGCCCACTATTAAGGAAGGTAACTCACGAGAACTAATGGGCCAACAATCTATGCCCAACAGGCCTAATTACATTGC
TTTTAGGGACAATTTTATTGGTCTAATGTATTACAACAGCACGGGTAATATGGGTGTTCTGGCGGGCCAAGCATCGCAGTTGAATGCTGTTGTAGA
TTTGCAAGACAGAAACACAGAGCTTTCATACCAGCTTTTGCTTGATTCCATTGGTGATAGAACCAGGTACTTTTCTATGTGGAATCAGGCTGTTGA
CAGCTATGATCCAGATGTTAGAATTATTGAAAATCATGGAACTGAAGATGAACTTCCAAATTACTGCTTTCCACTGGGAGGTGTGATTAATACAGA
GACTCTTACCAAGGTAAAACCTAAAACAGGTCAGGAAAATGGATGGGAAAAAGATGCTACAGAATTTTCAGATAAAAATGAAATAAGAGTTGGAAA
TAATTTTGCCATGGAAATCAATCTAAATGCCAACCTGTGGAGAAATTTCCTGTACTCCAACATAGCGCTGTATTTGCCCGACAAGCTAAAGTACAG
TCCTTCCAACGTAAAAATTTCTGATAACCCAAACACCTACGACTACATGAACAAGCGAGTGGTGGCTCCCGGGTTAGTGGACTGCTACATTAACCT
TGGAGCACGCTGGTCCCTTGACTATATGGACAACGTCAACCCATTTAACCACCACCGCAATGCTGGCCTGCGCTACCGCTCAATGTTGCTGGGCAA
TGGTCGCTATGTGCCCTTCCACATCCAGGTGCCTCAGAAGTTCTTTGCCATTAAAAACCTCCTTCTCCTGCCGGGCTCATACACCTACGAGTGGAA
CTTCAGGAAGGATGTTAACATGGTTCTGCAGAGCTCCCTAGGAAATGACCTAAGGGTTGACGGAGCCAGCATTAAGTTTGATAGCATTTGCCTTTA
CGCCACCTTCTTCCCCATGGCCCACAACACCGCCTCCACGCTTGAGGCCATGCTTAGAAACGACACCAACGACCAGTCCTTTAACGACTATCTCTC
CGCCGCCAACATGCTCTACCCTATACCCGCCAACGCTACCAACGTGCCCATATCCATCCCCTCCCGCAACTGGGCGGCTTTCCGCGGCTGGGCCTT
CACGCGCCTTAAGACTAAGGAAACCCCATCACTGGGCTCGGGCTACGACCCTTATTACACCTACTCTGGCTCTATACCCTACCTAGATGGAACCTT
TTACCTCAACCACACCTTTAAGAAGGTGGCCATTACCTTTGACTCTTCTGTCAGCTGGCCTGGCAATGACCGCCTGCTTACCCCCAACGAGTTTGA
AATTAAGCGCTCAGTTGACGGGGAGGGTTACAACGTTGCCCAGTGTAACATGACCAAAGACTGGTTCCTGGTACAAATGCTAGCTAACTACAACAT
TGGCTACCAGGGCTTCTATATCCCAGAGAGCTACAAGGACCGCATGTACTCCTTCTTTAGAAACTTCCAGCCCATGAGCCGTCAGGTGGTGGATGA
TACTAAATACAAGGACTACCAACAGGTGGGCATCCTACACCAACACAACAACTCTGGATTTGTTGGCTACCTTGCCCCCACCATGCGCGAAGGACA
GGCCTACCCTGCTAACTTCCCCTATCCGCTTATAGGCAAGACCGCAGTTGACAGCATTACCCAGAAAAAGTTTCTTTGCGATCGCACCCTTTGGCG
CATCCCATTCTCCAGTAACTTTATGTCCATGGGCGCACTCACAGACCTGGGCCAAAACCTTCTCTACGCCAACTCCGCCCACGCGCTAGACATGAC
TTTTGAGGTGGATCCCATGGACGAGCCCACCCTTCTTTATGTTTTGTTTGAAGTCTTTGACGTGGTCCGTGTGCACCGGCCGCACCGCGGCGTCAT
CGAAACCGTGTACCTGCGCACGCCCTTCTCGGCCGGCAACGCCACAACATAAAGAAGCAAGCAACATCAACAACAGCTGCCGCCATGGGCTCCAGT
GAGCAGGAACTGAAAGCCATTGTCAAAGATCTTGGTTGTGGGCCATATTTTTTGGGCACCTATGACAAGCGCTTTCCAGGCTTTGTTTCTCCACAC
AAGCTCGCCTGCGCCATAGTCAATACGGCCGGTCGCGAGACTGGGGGCGTACACTGGATGGCCTTTGCCTGGAACCCGCACTCAAAAACATGCTAC
CTCTTTGAGCCCTTTGGCTTTTCTGACCAGCGACTCAAGCAGGTTTACCAGTTTGAGTACGAGTCACTCCTGCGCCGTAGCGCCATTGCTTCTTCC
CCCGACCGCTGTATAACGCTGGAAAAGTCCACCCAAAGCGTACAGGGGCCCAACTCGGCCGCCTGTGGACTATTCTGCTGCATGTTTCTCCACGCC
TTTGCCAACTGGCCCCAAACTCCCATGGATCACAACCCCACCATGAACCTTATTACCGGGGTACCCAACTCCATGCTCAACAGTCCCCAGGTACAG
CCCACCCTGCGTCGCAACCAGGAACAGCTCTACAGCTTCCTGGAGCGCCACTCGCCCTACTTCCGCAGCCACAGTGCGCAGATTAGGAGCGCCACT
TCTTTTTGTCACTTGAAAAACATGTAAAAATAATGTACTAGAGACACTTTCAATAAAGGCAAATGCTTTTATTTGTACACTCTCGGGTGATTATTT
ACCCCCACCCTTGCCGTCTGCGCCGTTTAAAAATCAAAGGGGTTCTGCCGCGCATCGCTATGCGCCACTGGCAGGGACACGTTGCGATACTGGTGT
TTAGTGCTCCACTTAAACTCAGGCACAACCATCCGCGGCAGCTCGGTGAAGTTTTCACTCCACAGGCTGCGCACCATCACCAACGCGTTTAGCAGG
TCGGGCGCCGATATCTTGAAGTCGCAGTTGGGGCCTCCGCCCTGCGCGCGCGAGTTGCGATACACAGGGTTGCAGCACTGGAACACTATCAGCGCC
GGGTGGTGCACGCTGGCCAGCACGCTCTTGTCGGAGATCAGATCCGCGTCCAGGTCCTCCGCGTTGCTCAGGGCGAACGGAGTCAACTTTGGTAGC
TGCCTTCCCAAAAAGGGCGCGTGCCCAGGCTTTGAGTTGCACTCGCACCGTAGTGGCATCAAAAGGTGACCGTGCCCGGTCTGGGCGTTAGGATAC
AGCGCCTGCATAAAAGCCTTGATCTGCTTAAAAGCCACCTGAGCCTTTGCGCCTTCAGAGAAGAACATGCCGCAAGACTTGCCGGAAAACTGATTG
GCCGGACAGGCCGCGTCGTGCACGCAGCACCTTGCGTCGGTGTTGGAGATCTGCACCACATTTCGGCCCCACCGGTTCTTCACGATCTTGGCCTTG
CTAGACTGCTCCTTCAGCGCGCGCTGCCCGTTTTCGCTCGTCACATCCATTTCAATCACGTGCTCCTTATTTATCATAATGCTTCCGTGTAGACAC
TTAAGCTCGCCTTCGATCTCAGCGCAGCGGTGCAGCCACAACGCGCAGCCCGTGGGCTCGTGATGCTTGTAGGTCACCTCTGCAAACGACTGCAGG
TACGCCTGCAGGAATCGCCCCATCATCGTCACAAAGGTCTTGTTGCTGGTGAAGGTCAGCTGCAACCCGCGGTGCTCCTCGTTCAGCCAGGTCTTG
CATACGGCCGCCAGAGCTTCCACTTGGTCAGGCAGTAGTTTGAAGTTCGCCTTTAGATCGTTATCCACGTGGTACTTGTCCATCAGCGCGCGCGCA
GCCTCCATGCCCTTCTCCCACGCAGACACGATCGGCACACTCAGCGGGTTCATCACCGTAATTTCACTTTCCGCTTCGCTGGGCTCTTCCTCTTCC
TCTTGCGTCCGCATACCACGCGCCACTGGGTCGTCTTCATTCAGCCGCCGCACTGTGCGCTTACCTCCTTTGCCATGCTTGATTAGCACCGGTGGG
TTGCTGAAACCCACCATTTGTAGCGCCACATCTTCTCTTTCTTCCTCGCTGTCCACGATTACCTCTGGTGATGGCGGGCGCTCGGGCTTGGGAGAA
GGGCGCTTCTTTTTCTTCTTGGGCGCAATGGCCAAATCCGCCGCCGAGGTCGATGGCCGCGGGCTGGGTGTGCGCGGCACCAGCGCGTCTTGTGAT
GAGTCTTCCTCGTCCTCGGACTCGATACGCCGCCTCATCCGCTTTTTTGGGGGCGCCCGGGGAGGCGGCGGCGACGGGGACGGGGACGACACGTCC
TCCATGGTTGGGGGACGTCGCGCCGCACCGCGTCCGCGCTCGGGGGTGGTTTCGCGCTGCTCCTCTTCCCGACTGGCCATTTCCTTCTCCTATAGG
CAGAAAAAGATCATGGAGTCAGTCGAGAAGAAGGACAGCCTAACCGCCCCCTCTGAGTTCGCCACCACCGCCTCCACCGATGCCGCCAACGCGCCT
ACCACCTTCCCCGTCGAGGCACCCCCGCTTGAGGAGGAGGAAGTGATTATCGAGCAGGACCCAGGTTTTGTAAGCGAAGACGACGAGGACCGCTCA
GTACCAACAGAGGATAAAAAGCAAGACCAGGACAACGCAGAGGCAAACGAGGAACAAGTCGGGCGGGGGGACGAAAGGCATGGCGACTACCTAGAT
GTGGGAGACGACGTGCTGTTGAAGCATCTGCAGCGCCAGTGCGCCATTATCTGCGACGCGTTGCAAGAGCGCAGCGATGTGCCCCTCGCCATAGCG
GATGTCAGCCTTGCCTACGAACGCCACCTATTCTCACCGCGCGTACCCCCCAAACGCCAAGAAAACGGCACATGCGAGCCCAACCCGCGCCTCAAC
TTCTACCCCGTATTTGCCGTGCCAGAGGTGCTTGCCACCTATCACATCTTTTTCCAAAACTGCAAGATACCCCTATCCTGCCGTGCCAACCGCAGC
CGAGCGGACAAGCAGCTGGCCTTGCGGCAGGGCGCTGTCATACCTGATATCGCCTCGCTCAACGAAGTGCCAAAAATCTTTGAGGGTCTTGGACGC
GACGAGAAGCGCGCGGCAAACGCTCTGCAACAGGAAAACAGCGAAAATGAAAGTCACTCTGGAGTGTTGGTGGAACTCGAGGGTGACAACGCGCGC
CTAGCCGTACTAAAACGCAGCATCGAGGTCACCCACTTTGCCTACCCGGCACTTAACCTACCCCCCAAGGTCATGAGCACAGTCATGAGTGAGCTG
ATCGTGCGCCGTGCGCAGCCCCTGGAGAGGGATGCAAATTTGCAAGAACAAACAGAGGAGGGCCTACCCGCAGTTGGCGACGAGCAGCTAGCGCGC
TGGCTTCAAACGCGCGAGCCTGCCGACTTGGAGGAGCGACGCAAACTAATGATGGCCGCAGTGCTCGTTACCGTGGAGCTTGAGTGCATGCAGCGG
TTCTTTGCTGACCCGGAGATGCAGCGCAAGCTAGAGGAAACATTGCACTACACCTTTCGACAGGGCTACGTACGCCAGGCCTGCAAGATCTCCAAC
GTGGAGCTCTGCAACCTGGTCTCCTACCTTGGAATTTTGCACGAAAACCGCCTTGGGCAAAACGTGCTTCATTCCACGCTCAAGGGCGAGGCGCGC
CGCGACTACGTCCGCGACTGCGTTTACTTATTTCTATGCTACACCTGGCAGACGGCCATGGGCGTTTGGCAGCAGTGCTTGGAGGAGTGCAACCTC
AAGGAGCTGCAGAAACTGCTAAAGCAAAACTTGAAGGACCTATGGACGGCCTTCAACGAGCGCTCCGTGGCCGCGCACCTGGCGGACATCATTTTC
CCCGAACGCCTGCTTAAAACCCTGCAACAGGGTCTGCCAGACTTCACCAGTCAAAGCATGTTGCAGAACTTTAGGAACTTTATCCTAGAGCGCTCA
GGAATCTTGCCCGCCACCTGCTGTGCACTTCCTAGCGACTTTGTGCCCATTAAGTACCGCGAATGCCCTCCGCCGCTTTGGGGCCACTGCTACCTT
CTGCAGCTAGCCAACTACCTTGCCTACCACTCTGACATAATGGAAGACGTGAGCGGTGACGGTCTACTGGAGTGTCACTGTCGCTGCAACCTATGC
ACCCCGCACCGCTCCCTGGTTTGCAATTCGCAGCTGCTTAACGAAAGTCAAATTATCGGTACCTTTGAGCTGCAGGGTCCCTCGCCTGACGAAAAG
TCCGCGGCTCCGGGGTTGAAACTCACTCCGGGGCTGTGGACGTCGGCTTACCTTCGCAAATTTGTACCTGAGGACTACCACGCCCACGAGATTAGG
TTCTACGAAGACCAATCCCGCCCGCCAAATGCGGAGCTTACCGCCTGCGTCATTACCCAGGGCCACATTCTTGGCCAATTGCAAGCCATCAACAAA
GCCCGCCAAGAGTTTCTGCTACGAAAGGGACGGGGGGTTTACTTGGACCCCCAGTCCGGCGAGGAGCTCAACCCAATCCCCCCGCCGCCGCAGCCC
TATCAGCAGCAGCCGCGGGCCCTTGCTTCCCAGGATGGCACCCAAAAAGAAGCTGCAGCTGCCGCCGCCACCCACGGACGAGGAGGAATACTGGGA
CAGTCAGGCAGAGGAGGTTTTGGACGAGGAGGAGGAGGACATGATGGAAGACTGGGAGAGCCTAGACGAGGAAGCTTCCGAGGTCGAAGAGGTGTC
AGACGAAACACCGTCACCCTCGGTCGCATTCCCCTCGCCGGCGCCCCAGAAATCGGCAACCGGTTCCAGCATGGCTACAACCTCCGCTCCTCAGGC
GCCGCCGGCACTGCCCGTTCGCCGACCCAACCGTAGATGGGACACCACTGGAACCAGGGCCGGTAAGTCCAAGCAGCCGCCGCCGTTAGCCCAAGA
GCAACAACAGCGCCAAGGCTACCGCTCATGGCGCGGGCACAAGAACGCCATAGTTGCTTGCTTGCAAGACTGTGGGGGCAACATCTCCTTCGCCCG
CCGCTTTCTTCTCTACCATCACGGCGTGGCCTTCCCCCGTAACATCCTGCATTACTACCGTCATCTCTACAGCCCATACTGCACCGGCGGCAGCGG
CAGCGGCAGCAACAGCAGCGGCCACACAGAAGCAAAGGCGACCGGATAGCAAGACTCTGACAAAGCCCAAGAAATCCACAGCGGCGGCAGCAGCAG
GAGGAGGAGCGCTGCGTCTGGCGCCCAACGAACCCGTATCGACCCGCGAGCTTAGAAACAGGATTTTTCCCACTCTGTATGCTATATTTCAACAGA
GCAGGGGCCAAGAACAAGAGCTGAAAATAAAAAACAGGTCTCTGCGATCCCTCACCCGCAGCTGCCTGTATCACAAAAGCGAAGATCAGCTTCGGC
GCACGCTGGAAGACGCGGAGGCTCTCTTCAGTAAATACTGCGCGCTGACTCTTAAGGACTAGTTTCGCGCCCTTTCTCAAATTTAAGCGCGAAAAC
TACGTCATCTCCAGCGGCCACACCCGGCGCCAGCACCTGTCGTCAGCGCCATTATGAGCAAGGAAATTCCCACGCCCTACATGTGGAGTTACCAGC
CACAAATGGGACTTGCGGCTGGAGCTGCCCAAGACTACTCAACCCGAATAAACTACATGAGCGCGGGACCCCACATGATATCCCGGGTCAACGGAA
TCCGCGCCCACCGAAACCGAATTCTCTTGGAACAGGCGGCTATTACCACCACACCTCGTAATAACCTTAATCCCCGTAGTTGGCCCGCTGCCCTGG
TGTACCAGGAAAGTCCCGCTCCCACCACTGTGGTACTTCCCAGAGACGCCCAGGCCGAAGTTCAGATGACTAACTCAGGGGCGCAGCTTGCGGGCG
GCTTTCGTCACAGGGTGCGGTCGCCCGGGCAGGGTATAACTCACCTGACAATCAGAGGGCGAGGTATTCAGCTCAACGACGAGTCGGTGAGCTCCT
CGCTTGGTCTCCGTCCGGACGGGACATTTCAGATCGGCGGCGCCGGCCGTCCTTCATTCACGCCTCGTCAGGCAATCCTAACTCTGCAGACCTCGT
CCTCTGAGCCGCGCTCTGGAGGCATTGGAACTCTGCAATTTATTGAGGAGTTTGTGCCATCGGTCTACTTTAACCCCTTCTCGGGACCTCCCGGCC
ACTATCCGGATCAATTTATTCCTAACTTTGACGCGGTAAAGGACTCGGCGGACGGCTACGACTGAATGTTAAGTGGAGAGGCAGAGCAACTGCGCC
TGAAACACCTGGTCCACTGTCGCCGCCACAAGTGCTTTGCCCGCGACTCCGGTGAGTTTTGCTACTTTGAATTGCCCGAGGATCATATCGAGGGCC
CGGCGCACGGCGTCCGGCTTACCGCCCAGGGAGAGCTTGCCCGTAGCCTGATTCGGGAGTTTACCCAGCGCCCCCTGCTAGTTGAGCGGGACAGGG
GACCCTGTGTTCTCACTGTGATTTGCAACTGTCCTAACCTTGGATTACATCAAGATCCTCTAGTTAATACTAGTATTTAAATAATTCCCTGGCATT
ATGCCCAGTACATGACCTTATGGGACTTTCCTACTTGGCAGTACATCTACGTATTAGTCATCGCTATTACCATGGTGATGCGGTTTTGGCAGTACA
TCAATGGGCGTGGATAGCGGTTTGACTCACGGGGATTTCCAAGTCTCCACCCCATTGACGTCAATGGGAGTTTGTTTTGGCACCAAAATCAACGGG
ACTTTCCAAAATGTCGTAACAACTCCGCCCCATTGACGCAAATGGGCGGTAGGCGTGTACGGTGGGAGGTCTATATAAGCAGAGCTCGTTTAGTGA
ACCGTCAGATCGCCTGGAGACGCCATCCACGCTGTTTTGACCTCCATAGAAGACACCGGGACCGATCCAGCCTGGGGATCAGTCTTCGAGTCGAGG
ATCCCCTGCTCCAGAGATGACCGGCTCAACCATCGCGCCCACAACGGACTATCGCAACACCACTGCTACCGGACTAACATCTGCCCTAAATTTACC
CCAAGTTCATGCCTTTGTCAATGACTGGGCGAGCTTGGACATGTGGTGGTTTTCCATAGCGCTTATGTTTGTTTGCCTTATTATTATGTGGCTTAT
TTGTTGCCTAAAGCGCAGACGCGCCAGACCCCCCATCTATAGGCCTATCATTGTGCTCAACCCACACAATGAAAAAATTCATAGATTGGACGGTCT
GAAACCATGTTCTCTTCTTTTACAGTATGATTAAATGAGACATGATTCCAAGCTTGTCGACTTCGAGCAACTTGTTTATTGCAGCTTATAATGGTT
ACAAATAAAGCAATAGCATCACAAATTTCACAAATAAAGCATTTTTTTCACTGCATTCTAGTTGTGGTTTGTCCAAACTCATCAATGTATCTTATC
ATGTCTGGATCGTCTAGCATCGATTAATTAACTAGAGTACCCGGGGATCTTATTCCCTTTAACTAATAAAAAAAAATAATAAAGCATCACTTACTT
AAAATCAGTTAGCAAATTTCTGTCCAGTTTATTCAGCAGCACCTCCTTGCCCTCCTCCCAGCTCTGGTATTGCAGCTTCCTCCTGGCTGCAAACTT
TCTCCACAATCTAAATGGAATGTCAGTTTCCTCCTGTTCCTGTCCATCCGCACCCACTATCTTCATGTTGTTGCAGATGAAGCGCGCAAGACCGTC
TGAAGATACCTTCAACCCCGTGTATCCATATGACACGGAAACCGGTCCTCCAACTGTGCCTTTTCTTACTCCTCCCTTTGTATCCCCCAATGGGTT
TCAAGAGAGTCCCCCTGGGGTACTCTCTTTGCGCCTATCCGAACCTCTAGTTACCTCCAATGGCATGCTTGCGCTCAAAATGGGCAACGGCCTCTC
TCTGGACGAGGCCGGCAACCTTACCTCCCAAAATGTAACCACTGTGAGCCCACCTCTCAAAAAAACCAAGTCAAACATAAACCTGGAAATATCTGC
ACCCCTCACAGTTACCTCAGAAGCCCTAACTGTGGCTGCCGCCGCACCTCTAATGGTCGCGGGCAACACACTCACCATGCAATCACAGGCCCCGCT
AACCGTGCACGACTCCAAACTTAGCATTGCCACCCAAGGACCCCTCACAGTGTCAGAAGGAAAGCTAGCCCTGCAAACATCAGGCCCCCTCACCAC
CACCGATAGCAGTACCCTTACTATCACTGCCTCACCCCCTCTAACTACTGCCACTGGTAGCTTGGGCATTGACTTGAAAGAGCCCATTTATACACA
AAATGGAAAACTAGGACTAAAGTACGGGGCTCCTTTGCATGTAACAGACGACCTAAACACTTTGACCGTAGCAACTGGTCCAGGTGTGACTATTAA
TAATACTTCCTTGCAAACTAAAGTTACTGGAGCCTTGGGTTTTGATTCACAAGGCAATATGCAACTTAATGTAGCAGGAGGACTAAGGATTGATTC
TCAAAACAGACGCCTTATACTTGATGTTAGTTATCCGTTTGATGCTCAAAACCAACTAAATCTAAGACTAGGACAGGGCCCTCTTTTTATAAACTC
AGCCCACAACTTGGATATTAACTACAACAAAGGCCTTTACTTGTTTACAGCTTCAAACAATTCCAAAAAGCTTGAGGTTAACCTAAGCACTGCCAA
GGGGTTGATGTTTGACGCTACAGCCATAGCCATTAATGCAGGAGATGGGCTTGAATTTGGTTCACCTAATGCACCAAACACAAATCCCCTCAAAAC
AAAAATTGGCCATGGCCTAGAATTTGATTCAAACAAGGCTATGGTTCCTAAACTAGGAACTGGCCTTAGTTTTGACAGCACAGGTGCCATTACAGT
AGGAAACAAAAATAATGATAAGCTAACTTTGTGGACCACACCAGCTCCATCTCCTAACTGTAGACTAAATGCAGAGAAAGATGCTAAACTCACTTT
GGTCTTAACAAAATGTGGCAGTCAAATACTTGCTACAGTTTCAGTTTTGGCTGTTAAAGGCAGTTTGGCTCCAATATCTGGAACAGTTCAAAGTGC
TCATCTTATTATAAGATTTGACGAAAATGGAGTGCTACTAAACAATTCCTTCCTGGACCCAGAATATTGGAACTTTAGAAATGGAGATCTTACTGA
AGGCACAGCCTATACAAACGCTGTTGGATTTATGCCTAACCTATCAGCTTATCCAAAATCTCACGGTAAAACTGCCAAAAGTAACATTGTCAGTCA
AGTTTACTTAAACGGAGACAAAACTAAACCTGTAACACTAACCATTACACTAAACGGTACACAGGAAACAGGAGACACAACTCCAAGTGCATACTC
TATGTCATTTTCATGGGACTGGTCTGGCCACAACTACATTAATGAAATATTTGCCACATCCTCTTACACTTTTTCATACATTGCCCAAGAATAAAG
AATCGTTTGTGTTATGTTTCAACGTGTTTATTTTTCAATTGCAGAAAATTTCAAGTCATTTTTCATTCAGTAGTATAGCCCCACCACCACATAGCT
TATACAGATCACCGTACCTTAATCAAACTCACAGAACCCTAGTATTCAACCTGCCACCTCCCTCCCAACACACAGAGTACACAGTCCTTTCTCCCC
GGCTGGCCTTAAAAAGCATCATATCATGGGTAACAGACATATTCTTAGGTGTTATATTCCACACGGTTTCCTGTCGAGCCAAACGCTCATCAGTGA
TATTAATAAACTCCCCGGGCAGCTCACTTAAGTTCATGTCGCTGTCCAGCTGCTGAGCCACAGGCTGCTGTCCAACTTGCGGTTGCTTAACGGGCG
GCGAAGGAGAAGTCCACGCCTACATGGGGGTAGAGTCATAATCGTGCATCAGGATAGGGCGGTGGTGCTGCAGCAGCGCGCGAATAAACTGCTGCC
GCCGCCGCTCCGTCCTGCAGGAATACAACATGGCAGTGGTCTCCTCAGCGATGATTCGCACCGCCCGCAGCATAAGGCGCCTTGTCCTCCGGGCAC
AGCAGCGCACCCTGATCTCACTTAAATCAGCACAGTAACTGCAGCACAGCACCACAATATTGTTCAAAATCCCACAGTGCAAGGCGCTGTATCCAA
AGCTCATGGCGGGGACCACAGAACCCACGTGGCCATCATACCACAAGCGCAGGTAGATTAAGTGGCGACCCCTCATAAACACGCTGGACATAAACA
TTACCTCTTTTGGCATGTTGTAATTCACCACCTCCCGGTACCATATAAACCTCTGATTAAACATGGCGCCATCCACCACCATCCTAAACCAGCTGG
CCAAAACCTGCCCGCCGGCTATACACTGCAGGGAACCGGGACTGGAACAATGACAGTGGAGAGCCCAGGACTCGTAACCATGGATCATCATGCTCG
TCATGATATCAATGTTGGCACAACACAGGCACACGTGCATACACTTCCTCAGGATTACAAGCTCCTCCCGCGTTAGAACCATATCCCAGGGAACAA
CCCATTCCTGAATCAGCGTAAATCCCACACTGCAGGGAAGACCTCGCACGTAACTCACGTTGTGCATTGTCAAAGTGTTACATTCGGGCAGCAGCG
GATGATCCTCCAGTATGGTAGCGCGGGTTTCTGTCTCAAAAGGAGGTAGACGATCCCTACTGTACGGAGTGCGCCGAGACAACCGAGATCGTGTTG
GTCGTAGTGTCATGCCAAATGGAACGCCGGACGTAGTCATATTTCCTGAAGCAAAACCAGGTGCGGGCGTGACAAACAGATCTGCGTCTCCGGTCT
CGCCGCTTAGATCGCTCTGTGTAGTAGTTGTAGTATATCCACTCTCTCAAAGCATCCAGGCGCCCCCTGGCTTCGGGTTCTATGTAAACTCCTTCA
TGCGCCGCTGCCCTGATAACATCCACCACCGCAGAATAAGCCACACCCAGCCAACCTACACATTCGTTCTGCGAGTCACACACGGGAGGAGCGGGA
AGAGCTGGAAGAACCATGTTTTTTTTTTTATTCCAAAAGATTATCCAAAACCTCAAAATGAAGATCTATTAAGTGAACGCGCTCCCCTCCGGTGGC
GTGGTCAAACTCTACAGCCAAAGAACAGATAATGGCATTTGTAAGATGTTGCACAATGGCTTCCAAAAGGCAAACGGCCCTCACGTCCAAGTGGAC
GTAAAGGCTAAACCCTTCAGGGTGAATCTCCTCTATAAACATTCCAGCACCTTCAACCATGCCCAAATAATTCTCATCTCGCCACCTTCTCAATAT
ATCTCTAAGCAAATCCCGAATATTAAGTCCGGCCATTGTAAAAATCTGCTCCAGAGCGCCCTCCACCTTCAGCCTCAAGCAGCGAATCATGATTGC
AAAAATTCAGGTTCCTCACAGACCTGTATAAGATTCAAAAGCGGAACATTAACAAAAATACCGCGATCCCGTAGGTCCCTTCGCAGGGCCAGCTGA
ACATAATCGTGCAGGTCTGCACGGACCAGCGCGGCCACTTCCCCGCCAGGAACCTTGACAAAAGAACCCACACTGATTATGACACGCATACTCGGA
GCTATGCTAACCAGCGTAGCCCCGATGTAAGCTTTGTTGCATGGGCGGCGATATAAAATGCAAGGTGCTGCTCAAAAAATCAGGCAAAGCCTCGCG
CAAAAAAGAAAGCACATCGTAGTCATGCTCATGCAGATAAAGGCAGGTAAGCTCCGGAACCACCACAGAAAAAGACACCATTTTTCTCTCAAACAT
GTCTGCGGGTTTCTGCATAAACACAAAATAAAATAACAAAAAAACATTTAAACATTAGAAGCCTGTCTTACAACAGGAAAAACAACCCTTATAAGC
ATAAGACGGACTACGGCCATGCCGGCGTGACCGTAAAAAAACTGGTCACCGTGATTAAAAAGCACCACCGACAGCTCCTCGGTCATGTCCGGAGTC
ATAATGTAAGACTCGGTAAACACATCAGGTTGATTCATCGGTCAGTGCTAAAAAGCGACCGAAATAGCCCGGGGGAATACATACCCGCAGGCGTAG
AGACAACATTACAGCCCCCATAGGAGGTATAACAAAATTAATAGGAGAGAAAAACACATAAACACCTGAAAAACCCTCCTGCCTAGGCAAAATAGC
ACCCTCCCGCTCCAGAACAACATACAGCGCTTCACAGCGGCAGCCTAACAGTCAGCCTTACCAGTAAAAAAGAAAACCTATTAAAAAAACACCACT
CGACACGGCACCAGCTCAATCAGTCACAGTGTAAAAAAGGGCCAAGTGCAGAGCGAGTATATATAGGACTAAAAAATGACGTAACGGTTAAAGTCC
ACAAAAAACACCCAGAAAACCGCACGCGAACCTACGCCCAGAAACGAAAGCCAAAAAACCCACAACTTCCTCAAATCGTCACTTCCGTTTTCCCAC
GTTACGTAACTTCCCATTTTAAGAAAACTACAATTCCCAACACATACAAGTTACTCCGCCCTAAAACCTACGTCACCCGCCCCGTTCCCACGCCCC
GCGCCACGTCACAAACTCCACCCCCTCATTATCATATTGGCTTCAATCCAAAATAAGGTATATTATTGATGAT
Partial DNA sequence of Ad5-yCD/mutTK_{SR39}rep-ADP (base 1 → 5100) (E1 Region)

(SEQ ID NO. 2)

```
yCD sequence is italicized; glycine polylinker is bolded; mutTK_{SR39} sequence is regular text;
mutations in mutTK_{SR39} are indicated
                10 20 30 40 50 60 70 80 90
TCCCTTCCAGCTCTCTGCCCCTTTTGGATTGAAGCCAATATGATAATGAGGGGGTGGAGTTTGTGACGTGGCGCGGGGCGTGGGAACGGGGCGGGT
100
GACG
              110 120 130 140 150 160 170 180 190
TAGTAGTGTGGCGGAAGTGTGATGTTGCAAGTGTGGCGGAACACATGTAAGCGACGGATGTGGCAAAAGTGACGTTTTTGGTGTGCGCCGGTGTAC
200
ACAG
              210 220 230 240 250 260 270 280 290
GAAGTGACAATTTTCGCGCGGTTTTAGGCGGATGTTGTAGTAAATTTGGGCGTAACCGAGTAAGATTTGGCCATTTTCGCGGGAAAACTGAATAAG
300
AGGA
                                                                                                        BglII/BamHI
              310 320 330 340 350 360 370 380 390
AGTGAAATCTGAATAATTTTGTGTTACTCATAGCGCGTAATCTCTAGCATCGAAGATCCATTTGTCTAGGGCCGCGGGGACTTTGACCGTTTACGT
400
GGAG
                                                                                                                                                                        E1A TATA
              410 420 430 440 450 460 470 480 490
ACTCGCCCAGGTGTTTTTCTCAGGTGTTTTCCGCGTTCCGGGTCAAAGTTGGCGTTTTATTATTATAGTCAGCTGACGTGTAGTGTATTTATACCC
500
GGTG
                                +1 E1A mRNA E1A start
              510 520 530 540 550 560 570 580 590
AGTTCCTCAAGAGGCCACTCTTGAGTGCCAGCGAGTAGAGTTTTCTCCTCCGAGCCGCTCCGACACCGGGACTGAAAATGAGACATATTATCTGCC
600
ACGG
              610 620 630 640 650 660 670 680 690
AGGTGTTATTACCGAAGAAATGGCCGCCAGTCTTTTGGACCAGCTGATCGAAGAGGTACTGGCTGATAATCTTCCACCTCCTAGCCATTTTGAACC
700
ACCT
              710 720 730 740 750 760 770 780 790
ACCCTTCACGAACTGTATGATTTAGACGTGACGGCCCCCGAAGATCCCAACGAGGAGGCGGTTTCGCAGATTTTTCCCGACTCTGTAATGTTGGCG
800
GTGC
              810 820 830 840 850 860 870 880 890
AGGAAGGGATTGACTTACTCACTTTTCCGCCGGCGCCCGGTTCTCCGGAGCCGCCTCACCTTTCCCGGCAGCCCGAGCAGCCGGAGCAGAGAGCCT
900
TGGG
                                                                                                                                                                                        E1A
              910 920 930 940 950 960 970 980 990
TCCGGTTTCTATGCCAAACCTTGTACCGGAGGTGATCGATCTTACCTGCCACGAGGCTGGCTTTCCACCCAGTGACGACGAGGATGAAGAGGGTGA
SD
1000
GGAG
            1010 1020 1030 1040 1050 1060 1070 1080 1090
TTGTGTTAGATTATGTGGAGCACCCCGGGCACGGTTGCAGGTCTTGTCATTATCACCGGAGGAATACGGGGGACCCAGATATTATGTGTTCGCTT
1100
TGCT
                                                            E1A SD
            1110 1120 1130 1140 1150 1160 1170 1180 1190
ATATGAGGACCTGTGGCATGTTTGTCTACAGTAAGTGAAAATTATGGGCAGTGGGTGATAGAGTGGTGGGTTTGGTGTGGTAATTTTTTTTTTAAT
1200
TTTT
                                                                                        E1A SA
            1210 1220 1230 1240 1250 1260 1270 1280 1290
ACAGTTTTGTGGTTTAAAGAATTTTGTATTGTGATTTTTTTAAAAGGTCCTGTGTCTGAACCTGAGCCTGAGCCCGAGCCAGAACCGGAGCCTGCA
1300
AGAC
            1310 1320 1330 1340 1350 1360 1370 1380 1390
CTACCCGCCGTCCTAAAATGGCGCCTGCTATCCTGAGACGCCCGACATCACCTGTGTCTAGAGAATGCAATAGTAGTACGGATAGCTGTGACTCCG
1400
GTCC
            1410 1420 1430 1440 1450 1460 1470 1480 1490
TTCTAACACACCTCCTGAGATACACCCGGTGGTCCCGCTGTGCCCCATTAAACCAGTTGCCGTGAGAGTTGGTGGGCGTCGCCAGGCTGTGGAATG
1500
TATC
                                                                                                                        E1A stop
            1510 1520 1530 1540 1550 1560 1570 1580 1590
GAGGACTTGCTTAACGAGCCTGGGCAACCTTTGGACTTGAGCTGTAAACGCCCCAGGCCATAAGGTGTAAACCTGTGATTGCGTGTGTGGTTAACG
1600
CCTT
                                                        E1A polyadenylation E1A mRNA 3′ terminus
            1610 1620 1630 1640 1650 1660 1670 1680 1690
TGTTTGCTGAATGAGTTGATGTAAGTTTAATAAAGGGTGAGATAATGTTTAACTTGCATGGCGTGTTAAATGGGGCGGGGCTTAAAGGGTATATAA
1700
TGCG
                                      +1 E1B mRNA 19 kDa E1B start
            1710 1720 1730 1740 1750 1760 1770 1780 1790
CCGTGGGCTAATCTTGGTTACATCTGACCTCATGGAGGCTTGGGAGTGTTTGGAAGATTTTTCTGCTGTGCGTAACTTGCTGGAACAGAGCTCTAA
1800
CAGT
            1810 1820 1830 1840 1850 1860 1870 1880 1890
ACCTCTTGGTTTTGGAGGTTTCTGTGGGGCTCATCCCAGGCAAAGTTAGTCTGCAGAATTAAGGAGGATTACAAGTGGGAATTTGAAGAGCTTTTG
1900
AAAT
            1910 1920 1930 1940 1950 1960 1970 1980 1990
CCTGTGGTGAGCTGTTTGATTCTTTGAATCTGGGTCACCAGGCGCTTTTCCAAGAGAAGGTCATCAAGACTTTGGATTTTTCCACACCGGGGCGCG
2000
CTGC
                                                                        55 kDa E1B start
            2010 2020 2030 2040 2050 2060 2070 2080 2090
GGCTGCTGTTGCTTTTTTGAGTTTTATAAAGGATAAATGGAGCGAAGAAACCCATCTGAGCGGGGGGTACCTGCTGGATTTTCTGGCCATGCATCT
2100
GTGG
            2110 2120 2130 2140 2150 2160 2170 2180 2190
AGAGCGGTTGTGAGACACAAGAATCGCCTGCTACTGTTGTCTTCCGTCCGCCCGGCGATAATACCGACGGAGGAGCAGCAGCAGCAGCAGGAGGAA
2200
GCCA
                                                                                                  55 kDa premature stops
                                                                                                                      19 kDa E1B stop BglII
            2210 2220 2230 2240 2250 2260 2270 2280 2290
GGCGGCGGCGGCAGGAGCAGAGCCCATGGAACCCGAGAGCCGGCCTGGACCCTCGGGAATGAATGTTGTATAGGTGGCTTAACTGTATAGATCTAA
2300
TTCC
            2310 2320 2330 2340 2350 2360 2370 2380 2390
CTGGCATTATGCCCAGTACATGACCTTATGGGACTTTCCTACTTGGCAGTACATCTACGTATTAGTCATCGCTATTACCATGGTGATGCGGTTTTG
2400
GCAG
            2410 2420 2430 2440 2450 2460 2470 2480 2490
TACATCAATGGGCGTGGATAGCGGTTTGACTCACGGGGATTTCCAAGTCTCCACCCCATTGACGTCAATGGGAGTTTGTTTTGGCACCAAAATCAA
2500
CGGG
                                                                                                        CMV TATA
            2510 2520 2530 2540 2550 2560 2570 2580 2590
ACTTTCCAAAATGTCGTAACAACTCCGCCCCATTGACGCAAATGGGCGGTAGGCGTGTACGGTGGGAGGTCTATATAAGCAGAGCTCGTTTAGTGA
2600
ACCG
+1 CMV BamHI
            2610 2620 2630 2640 2650 2660 2670 2680 2690
TCAGATCGCCTGGAGACGCCATCCACGCTGTTTTGACCTCCATAGAAGACACCGGGACCGATCCAGCCTGGGGATCTTCGAGTCGAGGGATCCCTC
XhoI
2700
GAGC
        CD start
            2710 2720 2730 2740 2750 2760 2770 2780 2790
CACCATGGGGACAGGGGGAATGGCAAGCAAGTGGGATCAGAAGGGTATGGACATTGCCTATGAGGAGGCGGCCTTAGGTTACAAAGAGGGTGGTGT
2800
TCCT
            2810 2820 2830 2840 2850 2860 2870 2880 2890
ATTGGCGGATGTCTTATCAATAACAAAGACGGAAGTGTTCTCGGTCGTGGTCACAACATGAGATTTCAAAAGGGATCCGCCACACTACATGGTGAG
2900
ATCT
            2910 2920 2930 2940 2950 2960 2970 2980 2990
CCACTTTGGAAAACTGTGGGAGATTAGAGGGCAAAGTGTACAAAGATACCACTTTGTATACGACGCTGTCTCCATGCGACATGTGTACAGGTGCCA
3000
TCAT
            3010 3020 3030 3040 3050 3060 3070 3080 3090
CATGTATGGTATTCCACGCTGTGTTGTCGGTGAGAACGTTAATTTCAAAAGTAAGGGCGAGAAATATTTACAAACTAGAGGTCACGAGGTTGTTGT
3100
TGTT
                                                                                                                                                          end of yCD
            3110 3120 3130 3140 3150 3160 3170 3180 3190
GACGATGAGAGGTGTAAAAAGATCATGAAACAATTTATCGATGAAAGACCTCAGGATTGGTTTGAAGATATTGGTGAGGGTGGAGGGGGAGGCGGT
polylinker
3200
GGGG
          NheI TK start
            3210 3220 3230 3240 3250 3260 3270 3280 3290
GAGGTGCTAGCATGGCTTCGTACCCCTGCCATCAACACGCGTCTGCGTTCGACCAGGCTGCGCGTTCTCGCGGCCATAGCAACCGACGTACGGCGT
3300
TGCG
            3310 3320 3330 3340 3350 3360 3370 3380 3390
CCCTCGCCGGCAGCAAGAAGCCACGGAAGTCCGCCTGGAGCAGAAAATGCCCACGCTACTGCGGGTTTATATAGACGGTCCTCACGGGATGGGGAA
3400
AACC
            3410 3420 3430 3440 3450 3460 3470 3480 3490
ACCACCACGCAACTGCTGGTGGCCCTGGGTTCGCGCGACGATATCGTCTACGTACCCGAGCCGATGACTTACTGGCAGGTGCTGGGGGCTTCCGAG
3500
ACAA
            3510 3520 3530 3540 3550 3560 3570 3580 3590
TCGCGAACATCTACACCACACAACACCGCCTCGACCAGGGTGAGATATCGGCCGGGGACGCGGCGGTGGTAATGACAAGCGCCCAGATAACAATGG
3600
GCAT
                                                                                                                                                                    mutTK_{SR39}
            3610 3620 3630 3640 3650 3660 3670 3680 3690
GCCTTATGCCGTGACCGACGCCGTTCTGGCTCCTCATGTCGGGGGGGAGGCTGGGAGTTCACATGCCCCGCCCCCGGCCCTCACCATCTTCCTCGA
mutations
3700
CCGC
                        mUtTK_{SR39} mutations
            3710 3720 3730 3740 3750 3760 3770 3780 3790
CATCCCATCGCCTTCATGCTGTGCTACCCGGCCGCGCGGTACCTTATGGGCAGCATGACCCCCCAGGCCGTGCTGGCGTTCGTGGCCCTCATCCCG
3800
CCGA
            3810 3820 3830 3840 3850 3860 3870 3880 3890
CCTTGCCCGGCACAAACATCGTGTTGGGGGCCCTTCCGGAGGACAGACACATCGACCGCCTGGCCAAACGCCAGCGCCCCGGCGAGCGGCTTGACC
3900
TGGC
            3910 3920 3930 3940 3950 3960 3970 3980 3990
TATGCTGGCCGCGATTCGCCGCGTTTACGGGCTGCTTGCCAATACGGTGCGGTATCTGCAGGGCGGCGGGTCGTGGTGGGAGGATTGGGGACAGCT
4000
TTCG
            4010 4020 4030 4040 4050 4060 4070 4080 4090
GGGACGGCCGTGCCGCCCCAGGGTGCCGAGCCCCAGAGCAACGCGGGCCCACGACCCCATATCGGGGACACGTTATTTACCCTGTTTCGGGCCCCC
4100
GAGT
            4110 4120 4130 4140 4150 4160 4170 4180 4190
TGCTGGCCCCCAACGGCGACCTGTATAACGTGTTTGCCTGGGCCTTGGACGTCTTGGCCAAACGCCTCCGTCCCATGCACGTCTTTATCCTGGATT
4200
ACGA
            4210 4220 4230 4240 4250 4260 4270 4280 4290
CCAATCGCCCGCCGGCTGCCGGGACGCCCTGCTGCAACTTACCTCCGGGATGGTCCAGACCCACGTCACCACCCCAGGCTCCATACCGACGATCTG
4300
CGAC
                                                                              TK stop EcoRI HindIII
            4310 4320 4330 4340 4350 4360 4370 4380 4390
CTGGCGCGCACGTTTGCCCGGGAGATGGGGGAGGCTAACTGAAACACGGAAGAATTCAAGCTTGTCGACTTCGAGCAACTTGTTTATTGCAGCTTA
4400
TAAT
              SV40 polyadenylation SV40 3′ terminus
            4410 4420 4430 4440 4450 4460 4470 4480 4490
GGTTACAAATAAAGCAATAGCATCACAAATTTCACAAATAAAGCATTTTTTTCACTGCATTCTAGTTGTGGTTTGTCCAAACTCATCAATGTATCT
4500
TATC
                                              BglII BamHI/BglII
            4510 4520 4530 4540 4550 4560 4570 4580 4590
ATGTCTGGATCGTCTAGCATCGAAGATCTGGATCTGGGCGTGGTTAAGGGTGGGAAAGAATATATAAGGTGGGGGTCTTATGTAGTTTTGTATCTG
4600
TTTT
    E1B SA
            4610 4620 4630 4640 4650 4660 4670 4680 4690
GCAGCAGCCGCCGCCGCCATGAGCACCAACTCGTTTGATGGAAGCATTGTGAGCTCATATTTGACAACGCGCATGCCCCCATGGGCCGGGGTGCGT
4700
CAGA
            4710 4720 4730 4740 4750 4760 4770 4780 4790
ATGTGATGGGCTCCAGCATTGATGGTCGCCCCGTCCTGCCCGCAAACTCTACTACCTTGACCTACGAGACCGTGTCTGGAACGCCGTTGGAGACTG
4800
CAGC
            4810 4820 4830 4840 4850 4860 4870 4880 4890
CTCCGCCGCCGCTTCAGCCGCTGCAGCCACCGCCCGCGGGATTGTGACTGACTTTGCTTTCCTGAGCCCGCTTGCAAGCAGTGCAGCTTCCCGTTC
4900
ATCC
            4910 4920 4930 4940 4950 4960 4970 4980 4990
GCCCGCGATGACAAGTTGACGGCTCTTTTGGCACAATTGGATTCTTTGACCCGGGAACTTAATGTCGTTTCTCAGCAGCTGTTGGATCTGCGCCAG
5000
CAGG
                                                                                              E1B polyadenylation E1B mRNA 3′
            5010 5020 5030 5040 5050 5060 5070 5080 5090
TTTCTGCCCTGAAGGCTTCCTCCCCTCCCAATGCGGTTTAAAACATAAATAAAAAACCAGACTCTGTTTGGATTTGGATCAAGCAAGTGTCTTGCT
terminus
5100
GTCT
Partial DNA sequence of Ad5-yCD/mutTK_{SR39}rep-ADP (base 29,163 → 30,079) (E3 Region)
```

(SEQ ID NO. 3)

```
ADP sequence is bolded
SwaI
                10 20 30 40 50 60 70 80 90
ATTTAAATAATTCCCTGGCATTATGCCCAGTACATGACCTTATGGGACTTTCCTACTTGGCAGTACATCTACGTATTAGTCATCGCTATTACCATG
100
GTGA
              110 120 130 140 150 160 170 180 190
TGCGGTTTTGGCAGTACATCAATGGGCGTGGATAGCGGTTTGACTCACGGGGATTTCCAAGTCTCCACCCCATTGACGTCAATGGGAGTTTGTTTT
200
GGCA
                                                                                                                                                                              CMV TATA
              210 220 230 240 250 260 270 280 290
CCAAAATCAACGGGACTTTCCAAAATGTCGTAACAACTCCGCCCCATTGACGCAAATGGGCGGTAGGCGTGTACGGTGGGAGGTCTATATAAGCAG
300
AGCT
                            +1 CMV
              310 320 330 340 350 360 370 380 390
CGTTTAGTGAACCGTCAGATCGCCTGGAGACGCCATCCACGCTGTTTTGACCTCCATAGAAGACACCGGGACCGATCCAGCCTGGGGATCAGTCTT
400
CGAG
        BamHI ADP start
              410 420 430 440 450 460 470 480 490
TCGAGGATCCCCTGCTCCAGAGATGACCGGCTCAACCATCGCGCCCACAACGGACTATCGCAACACCACTGCTACCGGACTAACATCTGCCCTAAA
500
TTTA
              510 520 530 540 550 560 570 580 590
CCCCAAGTTCATGCCTTTGTCAATGACTGGGCGAGCTTGGACATGTGGTGGTTTTCCATAGCGCTTATGTTTGTTTGCCTTATTATTATGTGGCTT
600
ATTT
              610 620 630 640 650 660 670 680 690
GTTGCCTAAAGCGCAGACGCGCCAGACCCCCCATCTATAGGCCTATCATTGTGCTCAACCCACACAATGAAAAAATTCATAGATTGGACGGTCTGA
700
AACC
                                              ADP stop HindIII SV40
              710 720 730 740 750 760 770 780 790
ATGTTCTCTTCTTTTACAGTATGATTAAATGAGACATGATTCCAAGCTTGTCGACTTCGAGCAACTTGTTTATTGCAGCTTATAATGGTTACAAAT
poly A
800
AAAG
                      SV40 3′ terminus
              810 820 830 840 850 860 870 880 890
CAATAGCATCACAAATTTCACAAATAAAGCATTTTTTTCACTGCATTCTAGTTGTGGTTTGTCCAAACTCATCAATGTATCTTATCATGTCTGGAT
900
CGTC
                  PacI
              910
TAGCATCGATTAATTAA
DNA Sequence and Translation of yCD/mutTK_{SR39} Fusion Gene
```

(SEQ ID NO. 4)

```
yCD sequence is italicized; glycine polylinker is bolded; mutTK_{SR39} sequence is regular text;
mutations in mutTK_{SR39} are indicated
                                                      10 20 30
  M G T G G M A S K W D Q K G M D I A Y E E A A L G Y K E G G
ATGGGGACAGGGGGAATGGCAAGCAAGTGGGATCAGAAGGGTATGGACATTGCCTATGAGGAGGCGGCCTTAGGTTACAAAGAGGGTGGT
                10 20 30 40 50 60 70 80 90
                                                      40 50 60
  V P I G G C L I N N K D G S V L G R G H N M R F Q K G S A T
GTTCCTATTGGCGGATGTCTTATCAATAACAAAGACGGAAGTGTTCTCGGTCGTGGTCACAACATGAGATTTCAAAAGGGATCCGCCACA
              100 110 120 130 140 150 160 170 180
                                                      70 80 90
  L H G E I S T L E N C G R L E G K V Y K D T T L Y T T L S P
CTACATGGTGAGATCTCCACTTTGGAAAACTGTGGGAGATTAGAGGGCAAAGTGTACAAAGATACCACTTTGTATACGACGCTGTCTCCA
              190 200 210 220 230 240 250 260 270
                                                    100 110 120
  C D M C T G A I I M Y G I P R C V V G E N V N F K S K G E K
TGCGACATGTGTACAGGTGCCATCATCATGTATGGTATTCCACGCTGTGTTGTCGGTGAGAACGTTAATTTCAAAAGTAAGGGCGAGAAA
              280 290 300 310 320 330 340 350 360
                                                    130 140 150
  Y L Q T R G H E V V V V D D E R C K K I M K Q F I D E R P Q
TATTTACAAACTAGAGGTCACGAGGTTGTTGTTGTTGACGATGAGAGGTGTAAAAAGATCATGAAACAATTTATCGATGAAAGACCTCAG
              370 380 390 400 410 420 430 440 450
                                                    160 170 180
  D W F E D I G E G G G G G G G G G A S M A S Y P C H Q H A S
GATTGGTTTGAAGATATTGGTGAGGGTGGAGGGGGAGGCGGTGGGGGAGGTGCTAGCATGGCTTCGTACCCCTGCCATCAACACGCGTCT
              460 470 480 490 500 510 520 530 540
                                                    190 200 210
  A F D Q A A R S R G H S N R R T A L R P R R Q Q E A T E V R
GCGTTCGACCAGGCTGCGCGTTCTCGCGGCCATAGCAACCGACGTACGGCGTTGCGCCCTCGCCGGCAGCAAGAAGCCACGGAAGTCCGC
              550 560 570 580 590 600 610 620 630
                                                    220 230 240
  L E Q K M P T L L R V Y I D G P H G M G K T T T T Q L L V A
CTGGAGCAGAAAATGCCCACGCTACTGCGGGTTTATATAGACGGTCCTCACGGGATGGGGAAAACCACCACCACGCAACTGCTGGTGGCC
              640 650 660 670 680 690 700 710 720
                                                    250 260 270
  L G S R D D I V Y V P E P M T Y W Q V L G A S E T I A N I Y
CTGGGTTCGCGCGACGATATCGTCTACGTACCCGAGCCGATGACTTACTGGCAGGTGCTGGGGGCTTCCGAGACAATCGCGAACATCTAC
              730 740 750 760 770 780 790 800 810
                                                    280 290 300
  T T Q H R L D Q G E I S A G D A A V V M T S A Q I T M G M P
ACCACACAACACCGCCTCGACCAGGGTGAGATATCGGCCGGGGACGCGGCGGTGGTAATGACAAGCGCCCAGATAACAATGGGCATGCCT
              820 830 840 850 860 870 880 890 900
                                                    310 320 330
WT HSV-1 TK L I F
WT HSV-1 TK C A T
  Y A V T D A V L A P H V G G E A G S S H A P P P A L T I F L
TATGCCGTGACCGACGCCGTTCTGGCTCCTCATGTCGGGGGGGAGGCTGGGAGTTCACATGCCCCGCCCCCGGCCCTCACCATCTTCCTC
              910 920 930 940 950 960 970 980 990
                                                    340 350 360
WT HSV-1 TK A L
WT HSV-1 TK GC C C
  D R H P I A F M L C Y P A A R Y L M G S M T P Q A V L A F V
GACCGCCATCCCATCGCCTTCATGCTGTGCTACCCGGCCGCGCGGTACCTTATGGGCAGCATGACCCCCCAGGCCGTGCTGGCGTTCGTG
            1000 1010 1020 1030 1040 1050 1060 1070 1080
                                                    370 380 390
  A L I P P T L P G T N I V L G A L P E D R H I D R L A K R Q
GCCCTCATCCCGCCGACCTTGCCCGGCACAAACATCGTGTTGGGGGCCCTTCCGGAGGACAGACACATCGACCGCCTGGCCAAACGCCAG
            1090 1100 1110 1120 1130 1140 1150 1160 1170
                                                    400 410 420
  R P G E R L D L A M L A A I R R V Y G L L A N T V R Y L Q G
CGCCCCGGCGAGCGGCTTGACCTGGCTATGCTGGCCGCGATTCGCCGCGTTTACGGGCTGCTTGCCAATACGGTGCGGTATCTGCAGGGC
            1180 1190 1200 1210 1220 1230 1240 1250 1260
                                                    430 440 450
  G G S W W E D W G Q L S G T A V P P Q G A E P Q S N A G P R
GGCGGGTCGTGGTGGGAGGATTGGGGACAGCTTTCGGGGACGGCCGTGCCGCCCCAGGGTGCCGAGCCCCAGAGCAACGCGGGCCCACGA
            1270 1280 1290 1300 1310 1320 1330 1340 1350
                                                    460 470 480
  P H I G D T L F T L F R A P E L L A P N G D L Y N V F A W A
CCCCATATCGGGGACACGTTATTTACCCTGTTTCGGGCCCCCGAGTTGCTGGCCCCCAACGGCGACCTGTATAACGTGTTTGCCTGGGCC
            1360 1370 1380 1390 1400 1410 1420 1430 1440
                                                    490 500 510
  L D V L A K R L R R M H V E L L D Y D Q S P A G C R D A L L
TTGGACGTCTTGGCCAAACGCCTCCGTCCCATGCACGTCTTTATCCTGGATTACGACCAATCGCCCGCCGGCTGCCGGGACGCCCTGCTG
            1450 1460 1470 1480 1490 1500 1510 1520 1530
                                                    520 530 540
  Q L T S G M V Q T H V T T P G S I P T I C D L A R T F A R E
CAACTTACCTCCGGGATGGTCCAGACCCACGTCACCACCCCAGGCTCCATACCGACGATCTGCGACCTGGCGCGCACGTTTGCCCGGGAG
            1540 1550 1560 1570 1580 1590 1600 1610 1620
                        545
  M G E A N
ATGGGGGAGGCTAAC
            1630
DNA Sequence and Translation of ADP gene
```

(SEQ ID NO. 5)

```
                                                      10 20 30
  M T G S T I A P T T D Y R N T T A T G L T S A L N L P Q V H
ATGACCGGCTCAACCATCGCGCCCACAACGGACTATCGCAACACCACTGCTACCGGACTAACATCTGCCCTAAATTTACCCCAAGTTCAT
                10 20 30 40 50 60 70 80 90
                                                      40 50 60
  A F V N D W A S L D M W W F S I A L M F V C L I I M W L I C
GCCTTTGTCAATGACTGGGCGAGCTTGGACATGTGGTGGTTTTCCATAGCGCTTATGTTTGTTTGCCTTATTATTATGTGGCTTATTTGT
              100 110 120 130 140 150 160 170 180
                                                      70 80 90
  C L A R R R A R P P I Y R P I I V L N P H N E K I H R L D G
TGCCTAAAGCGCAGACGCGCCAGACCCCCCATCTATAGGCCTATCATTGTGCTCAACCCACACAATGAAAAAATTCATAGATTGGACGGT
              190 200 210 220 230 240 250 260 270
                                                    100
  L K P C S L L L Q Y D
CTGAAACCATGTTCTCTTCTTTTACAGTATGAT
              280 290 300
```

What is claimed is:

1. An isolated polynucleotide comprising a nucleotide sequence of a yeast cytosine deaminase/mutant SR 39 herpes simplex virus type 1 thymidine kinase fusion gene.

2. An isolated polypeptide comprising an amino acid sequence encoded by the polynucleotide of claim 1, which converts prodrugs, 5 fluorocytosine and ganciclovir, into active chemotherapeutic agents.

3. A recombinant adenovirus comprising the polynucleotide according to claim 1.

4. The recombinant adenovirus according to claim 3, further comprising an adenovirus type 5 adenovirus death protein gene.

5. The recombinant adenovirus according to claim 3, wherein the adenovirus is a replication-competent, type 5 adenovirus.

6. The polynucleotide according to claim 1, further comprising an adenovirus type 5 adenovirus death protein gene.

7. The polynucleotide according to claim 1, which comprises the nucleotide sequence of SEQ ID NO. 4.

8. The polynucleotide according to claim 4, wherein the adenovirus type 5 adenovirus death protein gene comprises the nucleotide sequence of SEQ ID NO. 5.

9. The polypeptide according to claim 2, which comprises an amino acid sequence of SEQ ID NO. 4.

10. The recombinant adenovirus according to claim 4, which comprises the nucleotide sequence of SEQ ID NO. 1.

11. A pharmaceutical composition comprising the recombinant adenovirus according to claim 3, and a pharmaceutically acceptable carrier.

12. A pharmaceutical composition comprising the recombinant adenovirus according to claim 4, and a pharmaceutically acceptable carrier.

13. A method of treating a mammalian patient having a malignancy, said method comprising administering to the patient the pharmaceutical composition according to claim 11.

14. A method of treating a mammalian patient having a malignancy, said method comprising administering to the patient the pharmaceutical composition according to claim 12.

15. The method according to claim 13, wherein the pharmaceutical composition is administered locally to a tumor site.

16. The method according to claim 13, wherein the pharmaceutical composition is administered by direct injection to a tumor.

17. The method according to claim 13, wherein the pharmaceutical composition is administered by intravenous injection.

18. The method according to claim 13, wherein the administration is performed at two or more separate times.

19. The method according to claim 14, wherein the pharmaceutical composition is administered locally to a tumor site.

20. The method according to claim 14, wherein the pharmaceutical composition is administered by direct injection to a tumor.

21. The method according to claim 14, wherein the pharmaceutical composition is administered by intravenous injection.

22. The method according to claim 14, wherein the administration is performed at two or more separate times.

23. The method according to claim 14, further comprising administering (a) 5-fluorocytosine and/or (b) ganciclovir or derivatives thereof to the patient.

24. The method according to claim 23, further comprising treating the patient with radiation therapy.

25. A method of treating a mammalian patient having a solid tumor, wherein cells comprising said tumor are capable of infection by an adenovirus, said method comprising:
treating the patient with the recombinant adenovirus according to claim 4,
administering (a) 5-fluorocytosine and/or (b) ganciclovir or derivatives thereof to the patient, and
treating the patient with radiation therapy.

26. A method of converting 5 fluorocytosine and/or ganciclovir into active chemotherapeutic agents comprising contacting the polypeptide according to claim 2 with 5 fluorocytosine and/or ganciclovir.
